(19) United States
(12) Patent Application Publication
(10) Pub. No.: US 2024/0368291 A1
(43) Pub. Date: Nov. 7, 2024

(54) ACTRII ANTIBODY TREATMENTS

(71) Applicant: Versanis Bio, Inc., Oakland, CA (US)

(72) Inventors: Lloyd B. KLICKSTEIN, Newton, MA (US); Matthias MACHACEK, Allschwil (CH)

(21) Appl. No.: 18/776,590

(22) Filed: Jul. 18, 2024

Related U.S. Application Data
(63) Continuation of application No. PCT/US23/60932, filed on Jan. 19, 2023.
(60) Provisional application No. 63/373,676, filed on Aug. 26, 2022.
(60) Provisional application No. 63/301,011, filed on Jan. 19, 2022.

(51) Int. Cl. C07K 16/28

(52) U.S. Cl. CPC C07K 16/2863

(57) ABSTRACT

Provided herein are ActRII antibody dosage regimens, for the treatment of metabolic disorders, including obesity. In some embodiments, the dosage regimen is preceded by the administration of a loading dose of an ActRII antibody and can further optimize the efficacy of the treatment.

### CROSS REFERENCE TO RELATED APPLICATIONS

This application claims priority to U.S. Provisional Patent Application Nos. 63/301,011 filed Jan. 19, 2022, and 63/373,676 filed Aug. 26, 2022, the contents of which are incorporated by reference in their entirety.

### INCORPORATION BY REFERENCE OF SEQUENCE LISTING

The contents of the electronic sequence listing (VRNS_008_02WO_SeqList_ST26.xml; Size: 10,565 bytes; and Date of Creation: Jan. 18, 2023) are herein incorporated by reference in its entirety.

### BACKGROUND

Treatment of metabolic disorders such as obesity remains elusive as the regulation of metabolism and fat storage involves complex biological feedback systems. Even when a safe and effective drug is identified as capable of treating metabolic disorders, the dosing paradigms may be further optimized for effective and efficient dosing in overweight and obese patient populations, and for populations presenting with obesity-related comorbidities, who often require long term treatment. Therefore, a need exists for optimized dosing paradigms for the treatment of metabolic disorders, including obesity, and accordingly are provided herein.

### SUMMARY

The disclosure relates to the dosage and administration of ActRII pathway agents, for example ActRII antibodies, for the treatment of metabolic disorders. Treatments with an ActRII antibody can improve metabolic dysfunction, including reducing fat mass, increasing lean mass, and/or improving glycemic control. In some embodiments, provided herein are ActRII antibody treatments comprising ActRII antibody dosage regimens for the treatment of a metabolic disorder. In some embodiments, an ActRII antibody treatment comprises one or more loading doses of an ActRII antibody administered before the start of an ActRII antibody dosage regimen.

In one aspect, provided herein are ActRII antibody dosage regimens comprising administration of an ActRII antibody about once every 8 weeks to about once every 16 weeks, in a dose of about 3 mg/kg to about 50 mg/kg, wherein the ActRII antibody dosage regimen is administered intravenously. In some embodiments, the ActRII antibody dosage regimen follows the administration of an ActRII antibody loading dose.

In some embodiments, an ActRII antibody dosage regimen and/or an ActRII antibody loading dose comprises administration of an ActRII antibody comprising the amino acid sequence of SEQ ID NOS: 1-6. In some embodiments, an ActRII antibody dosage regimen and/or an ActRII antibody loading dose comprises administration of an ActRII antibody comprising: the amino acid sequence of SEQ ID NO: 7, or a sequence with sequence identity of at least 90% thereto, and comprising the amino acid sequence of SEQ ID NO: 8, or a sequence with sequence identity of at least 90% thereto. In some embodiments, an ActRII antibody dosage regimen and/or an ActRII antibody loading dose comprises administration of an ActRII antibody comprising: the amino acid sequence of SEQ ID NO: 9, or a sequence with sequence identity of at least 90% thereto, and comprising the amino acid sequence of SEQ ID NO: 10, or a sequence with sequence identity of at least 90% thereto. In some embodiments, an ActRII antibody dosage regimen and/or an ActRII antibody loading dose comprises administration of an ActRII antibody specific for ActRIIA and ActRIIB.

In some embodiments, an ActRII antibody treatment provided herein treats a metabolic disorder selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity. In some embodiments, the monogenetic disorder associated with obesity is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B. In some embodiments, the metabolic disorder is Prader-Willi syndrome. In some embodiments, the diabetes is Type I diabetes or Type II diabetes.

In some embodiments, an ActRII antibody treatment provided herein treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidemia, hypertension, heart failure, coronary heart disease, stroke, and/or gallstones.

In some embodiments the ActRII antibody treatment reduces body weight in the subject. In some embodiments the ActRII antibody treatment reduces fat mass in the subject. In some embodiments the ActRII antibody treatment increases lean mass in the subject. In some embodiments the ActRII antibody treatment reduces fat mass and increases lean mass in the subject. In some embodiments the ActRII antibody treatment reduces fat mass and maintains lean mass in the subject. In some embodiments the ActRII antibody treatment reduces waist circumference in the subject. In some embodiments the ActRII antibody treatment reduces liver and/or non-liver fat mass in the subject. In some embodiments the ActRII antibody treatment improves glycemic control in the subject.

In some embodiments the efficacy of the ActRII antibody treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

In some embodiments, the efficacy of the ActRII antibody treatment is measured by the C_{trough} in a sample from the subject. In some embodiments, the ActRII antibody treatment is efficacious when the C_{trough} of the sample from the subject is at least about 500%, about 400%, about 300%, about 200%, about 100%, about 90%, about 80%, or about 75% of a desired “C_{trough}”. In some embodiments, the desired “C_{trough}” of the ActRII antibody treatment is 10 μg/ml. In some embodiments, the subject of the ActRII antibody treatment is human.

In another aspect, provided herein is an ActRII antibody treatment comprising an ActRII antibody loading dose which is followed by an ActRII antibody dosage regimen.

In some embodiments, the ActRII antibody loading dose is about 3 mg/kg to about 50 mg/kg. In some embodiments, the ActRII antibody dosage regimen starts about 1 day, about 2 days, about 3 days, about 4 days, about 5 days, about 6 days, about 1 week, about 2 weeks, about 3 weeks, about 4 weeks, or about 5 weeks after the loading dose of the ActRII antibody. In some embodiments, the ActRII antibody dosage regimen comprises administration of an ActRII antibody in a dose of about 3 mg/kg to about 50 mg/kg. In some embodiments, the ActRII antibody dosage regimen comprises administration of an ActRII antibody about every 4 weeks, about every 8 weeks, about every 12 weeks, or about every 16 weeks. In some embodiments, the ActRII antibody loading dose and the ActRII antibody of the dosage regimen are administered intravenously.

In some embodiments, the ActRII antibody of the loading dose and/or the ActRII antibody dosage regimen comprises the amino acid sequence of SEQ ID NOS: 1-6. In some embodiments, the ActRII antibody of the loading dose and/or the ActRII antibody dosage regimen comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto. In some embodiments, the loading dose of the ActRII antibody and/or the ActRII antibody dosage regimen comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto. In some embodiments, the ActRII antibody of the loading dose and/or the ActRII antibody dosage regimen is specific for ActRIIA and ActRIIB.

In some embodiments, the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity. In some embodiments, the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B. In some embodiments, the metabolic disorder is Prader-Willi syndrome. In some embodiments, the diabetes is Type I diabetes or Type II diabetes.

In some embodiments, an ActRII antibody treatment provided herein treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

In some embodiments, the ActRII antibody treatment reduces body weight in the subject. In some embodiments, the ActRII antibody treatment reduces fat mass in the subject. In some embodiments, the ActRII antibody treatment increases lean mass in the subject. In some embodiments, the ActRII antibody treatment reduces fat mass and increases lean mass in the subject. In some embodiments, the ActRII antibody treatment reduces fat mass and maintains lean mass in the subject. In some embodiments, the ActRII antibody treatment reduces waist circumference in the subject. In some embodiments, the ActRII antibody treatment reduces liver and/or non-liver fat mass in the subject. In some embodiments, the ActRII antibody treatment improves glycemic control in the subject.

In some embodiments, the efficacy of the ActRII antibody treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

In some embodiments, the efficacy of the ActRII antibody treatment is measured by the C_{trough} in a sample from the subject. In some embodiments, the ActRII antibody treatment is efficacious when the C_{trough} of the sample from the subject is at least about 500%, about 400%, about 300%, about 200%, about 100%, about 90%, about 80%, or about 75% of a desired “C_{trough}”. In some embodiments, the desired “C_{trough}” of the ActRII antibody treatment is 10 μg/ml. In some embodiments, the subject of the ActRII antibody treatment is human.

### BRIEF DESCRIPTION OF THE DRAWINGS

FIG. 1 is a plot of individual patient fat body mass in kilograms (kg) versus lean body mass (kg) in the 7 clinical studies assessed, as described in the Examples.

FIG. 2 is a plot of individual patient age (years) versus lean body mass (kg) in the same 7 clinical studies assessed.

FIG. 3 is a plot of individual patient age (years) versus fat body mass (kg) in the same 7 clinical studies assessed.

FIG. 4 is a plot of individual patient age (years) versus body weight (kg) in the same 7 clinical studies assessed.

FIG. 5 is a pair of graphs showing (left) bimagrumab treated patients for whom there was an association between an increase in lean body mass (LBM) and a decrease in fat body mass (FBM), and (right) for the placebo group for whom there was no association between change in FBM and LBM.

FIG. 6 is a schematic of the pharmacokinetic and pharmacodynamic (PK/PD) bimagrumab dosage model.

FIG. 7 is a pair of graphs showing the observed versus model predicted bimagrumab exposure (PK) concentrations for the clinical study population (left) and individual patients (right).

FIG. 8 is a pair of graphs showing the observed versus predicted bimagrumab exposure (PK) concentrations of FIG. 7 on a log-log scale for the clinical study population (left) and individual patients (right).

FIG. 9 is a plot showing the predicted median with 90% confidence interval (area) and observed median for bimagrumab concentration exposure (PK) over time for all studies.

FIG. 10 is three graphs by patient weight (lowest on the left to highest on the right in kilograms) showing the predicted median with 90% confidence interval (area) and observed median for bimagrumab concentration exposure (PK) over time.

FIG. 11 is a pair of graphs showing observed versus predicted fat body mass (FBM) for the clinical study population (left) and individual patients (right).

FIG. 12 is three graphs showing the predicted median with 90% confidence interval (area) and observed median of fat body mass (FBM) by body weight groups (lowest on the left to highest on the right in kilograms).

FIG. 13 is a pair of graphs showing observed versus predicted lean body mass (LBM) for the clinical study population (left) and individual patients (right).

FIG. 14 is three graphs showing the predicted median with 90% confidence interval (area) and observed median of lean body mass (LBM) by body weight groups (lowest on the left to highest on the right in kilograms).

FIG. 15 is a plot showing the predicted median bimagrumab serum concentration (% relative to baseline) for different bimagrumab dosage regimens.

FIG. 16 is a plot showing the predicted median effect on FBM and LBM (% relative to baseline) for different bimagrumab dosage regimens.

FIG. 17 is a plot showing the predicted median effect on the sum of FBM and LBM (% relative to baseline) for different bimagrumab dosage regimens.

FIG. 18 is a plot showing the predicted median bimagrumab serum concentration (% relative to baseline) for different bimagrumab dosage regimens with and without a loading dose. The dosage regimens with a loading dose start 4 weeks after the administration of the loading dose (at week 4).

FIG. 19 is a plot showing the predicted median effect on FBM and LBM (% relative to baseline) for different bimagrumab dosage regimens with and without a loading dose. The dosage regimens with a loading dose start 4 weeks after the administration of the loading dose (at week 4).

FIG. 20 is a plot showing the predicted median effect on the sum of FBM and LBM (% relative to baseline) for different bimagrumab dosage regimens.

FIG. 21 is boxplots showing the predicted response (% relative to baseline) of fat body mass (FBM) and lean body mass (LBM) at weeks 24, 36, and 48 for different bimagrumab dosage regimens with and without a loading dose. The dosage regimens with a loading dose start 4 weeks after the administration of the loading dose (at week 4).

FIG. 22 is 2 graphs showing the effect of body weight on concentration-time profiles for the bimagrumab antibody treatments which include a loading dose at week 0, prior to the dosage regimen starting at week 4.

FIG. 23 is 2 graphs showing the effect of body weight on fat body mass (FBM)-time profiles for the bimagrumab antibody treatments which include a loading dose at week 0, prior to the dosage regimen starting at week 4.

FIG. 24 is a graph showing the predicted elimination of fat body mass (FBM half-life) relative to bimagrumab serum concentration. It is predicted that a loading dose helps maintain the serum concentration of an ActRII antibody above the saturation concentration of the ActRII receptor.

FIG. 25 is a schematic of an exemplary clinical study designed to assess the efficacy of bimagrumab antibody treatments administered every 12 weeks, including a loading dose at week 0, prior to the dosage regimen starting at week 4.

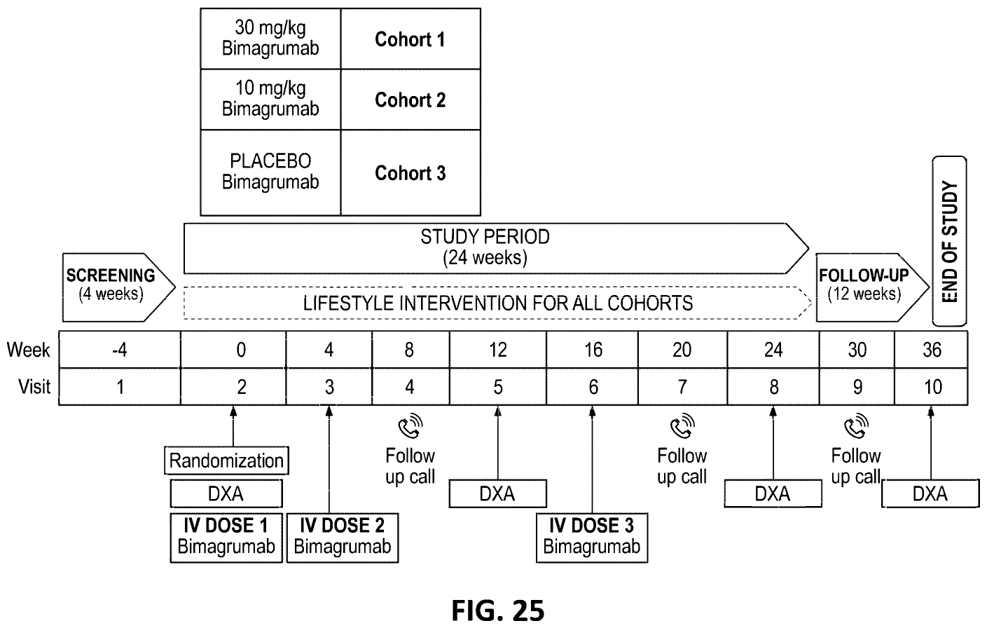

### DETAILED DESCRIPTION

Provided herein are dosing regimens of ActRII pathway agents, for example ActRII antibodies, for the treatment of metabolic disorders. The treatments with an ActRII antibody can improve metabolic dysfunction, including reducing fat mass, increasing lean mass, and/or improving glycemic control. In some embodiments, an ActRII antibody treatment comprises a dosage regimen, wherein the ActRII antibody is administered about once every 8 weeks to about once every 16 weeks, e.g., about once every 12 weeks, in a dose of about 3-50 mg/kg, e.g., about 30 mg/kg, and wherein the dose is administered intravenously. In some embodiments, an ActRII antibody treatment includes the use of one or more loading doses in addition to a dosage regimen.

### I. Definitions

Unless otherwise defined herein, scientific and technical terms used herein shall have the meanings that are commonly understood by those of ordinary skill in the art. Generally, nomenclature used in connection with, and techniques of, chemistry, molecular biology, cell biology, immunology, pharmacology, and protein chemistry, described herein, are those well-known and commonly used in the art.

It must be noted that, as used herein and in the appended claims, the singular forms “a,” “and,” and “the” include plural referents unless the context clearly dictates otherwise. Thus, for example, reference to “an agent” refers to one or mixtures of such candidates, and reference to “a method” includes reference to equivalent steps and methods known to those skilled in the art, and so forth.

As used herein, the term “approximately” or “about,” as applied to one or more values of interest, refers to a value that is similar in magnitude and/or within a similar range to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

As used herein, the terms “polypeptide,” “peptide,” and “protein” refer to polymers of amino acids of any length. The terms also encompass an amino acid polymer that has been modified; for example, to include disulfide bond formation, glycosylation, lipidation, phosphorylation, or conjugation with a labeling component.

As used herein, the terms “identity” and “identical” refer, with respect to a polypeptide sequence-of-interest, to the percentage of exact matching residues in an alignment of that the sequence-of-interest to a reference sequence, such as an alignment generated by the BLAST algorithm. Identity is calculated, unless specified otherwise, across the full length of the reference sequence. Thus a sequence-of-interest “shares at least x % identity to” a reference sequence if, when the reference sequence is aligned (as a query sequence) is aligned to the sequence-of-interest (as subject sequence), at least x % (rounded down) of the residues in the subject sequence are aligned as an exact match to a corresponding residue in the query sequence, the denominator being the full length of the reference sequence plus the lengths of any gaps inserted into the reference sequence by alignment of the reference sequence to the sequence-of-interest. Where the subject sequence has variable positions (e.g., residues denoted X), an alignment to any residue in the query sequence is counted as a match. Sequence alignments may be performed using the NCBI Blast service (BLAST+ version 2.12.0) or another program giving the same results.

As used herein, “antibody” includes reference to an immunoglobulin molecule immunologically reactive with a particular antigen, including both polyclonal and monoclonal antibodies. The term includes humanized antibodies, chimeric antibodies e.g., murine variable region with a human constant region) and conjugated antibodies. The term “antibody” also includes antigen binding forms of antibodies, including fragments that retain antigen-binding capability (e.g., Fab′, F(ab′)₂, Fab, single chain variable fragments (scFv) containing VH and VL sequences linked together in one chain, and single chain antibody fragments (scAb). The term antibody also includes bivalent or bispecific molecules, diabodies, triabodies, and tetrabodies.

The terms “treatment”, “treating” and the like are used herein to generally mean obtaining a desired pharmacologic and/or physiologic effect with a therapeutic agent. The effect may be prophylactic in terms of completely or partially preventing a disease or symptom thereof, e.g., reducing the likelihood that the disease or symptom thereof occurs in the subject, and/or may be therapeutic in terms of completely or partially reducing a symptom, or a partial or complete cure for a disease and/or adverse effect attributable to the disease. “Treatment” as used herein covers any treatment of a disease in a mammal, and includes: (a) preventing the disease from occurring in a subject which may be predisposed to the disease but has not yet been diagnosed as having it; (b) inhibiting or slowing the onset or development of the disease; or (c) relieving the disease, e.g., causing regression of the disease or symptoms associated with the disease. The therapeutic agent may be administered before, during or after the onset of disease. The treatment of ongoing disease, where the treatment stabilizes or reduces the undesirable clinical symptoms of the patient, may be of particular interest. In some embodiments, treatment is performed prior to complete loss of function in the affected tissues. In some embodiments, the subject's treatment will be administered during the symptomatic stage of the disease, and in some embodiments, after the symptomatic stage of the disease.

The term “dose,” as used herein, refers to the amount of therapeutic agent administered to a subject to achieve a therapeutic objective.

The term “loading dose” refers to one or more doses of a therapeutic agent that are administered in addition to a dosage regimen. As used herein a “loading dose” may refer to one or more doses of a therapeutic agent which are the same concentration, a lower concentration, or a higher concentration than the doses of a therapeutic agent administered as part of a dosage regimen. In some embodiments, a loading dose is administered prior to the commencement of a round of a dosing regimen.

The term “dosage” as used herein, refers to the amount, number, and frequency of doses over the course of the treatment.

The term “dosage regimen” as used herein refers to the repeated, regular administration of a therapeutic agent. The dosage regimen as used herein does not include a loading dose, which may, in some embodiments, be administered in addition, e.g., prior, to a round of a dosage regimen.

The terms “antibody treatment,” or “ActRII treatment” as used herein refer to a treatment comprising a dosage regimen. In some embodiments, the terms refer to the dosage regimen and one or more loading doses.

The terms “individual,” “subject,” and “patient” are used interchangeably herein and refer to any subject for whom treatment is desired. The subject may be a mammalian subject. Mammalian subjects include, e. g., humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

A metabolic disorder as used herein refers to a disorder affecting dysregulation of mammalian metabolism, including but not limited to: obesity, diabetes (Type I and II), metabolic syndrome, anti-psychotic drug-associated obesity, glucocorticoid-induced obesity, hypothalamic obesity associated with craniopharyngioma, and complex and monogenetic disorders associated with obesity. The monogenetic disorders associated with obesity in humans, may include but are not limited to Bardet-Biedl syndrome, and a disorder arising from a mutation in one or more of the following genes ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B, or combinations thereof. A metabolic disorder may also be associated with a complex genetic disorder, e.g., Prader-Willi syndrome.

As used herein, Body Mass Index or “BMI” is calculated as weight in kilograms (kg) divided by height in meters squared (m²), rounded to one decimal place. As used herein, “obesity” in adult humans is defined as a BMI greater than or equal to 30 kg/m². “Obesity” in human youth is defined as a BMI greater than or equal to the age- and sex-specific 95th percentile of the 2000 CDC growth charts. The term “overweight” is defined as a BMI of greater than or equal to 25 and less than 30.

The terms “obesity related co-morbidity” and “obesity related condition” may be used interchangeably and refer to a health condition depending from the obesity of the subject. In some embodiments, an obesity related co-morbidity or condition increases the mortality risk of the subject. Obesity related co-morbidities include but are not limited to: high blood pressure (hypertension), high LDL cholesterol, low HDL cholesterol, high levels of triglycerides (dyslipidemia), Type 2 diabetes, coronary heart disease, cardiometabolic disease, cardiometabolic syndrome, non-alcoholic fatty liver disease, stroke, gallbladder disease, osteoarthritis, sleep apnea, breathing problems, cancer, lower quality of life, mental illness such as clinical depression, anxiety, and other mental disorders, and body pain and difficulty with physical functioning. Obesity related cancers include but are not limited to: gastrointestinal cancers including but not limited to adenocarcinoma of the esophagus, stomach cancer, colon, and rectal cancers; breast cancer in post-menopausal women, uterine cancer, gallbladder cancer, kidney cancer, liver cancer, ovarian cancer, pancreatic cancer, thyroid cancer, meningioma, and multiple myeloma.

“Lean mass” is defined as total body mass of a subject minus the fat mass of the subject. Lean mass and fat mass may be measured by, for example, bioelectrical impedance analysis (BIA) or dual X-ray absorptiometry (DXA).

### II. ActRII Pathway Modulators

### ActRII Antibodies

Activin receptor II B (ActRIIB) is a receptor for myostatin, activin, and bone morphogenetic proteins (BMPs). The interaction between myostatin and this receptor regulates the inhibition of skeletal muscle differentiation via the Smad-dependent pathway. It is thought that by inhibiting or preventing myostatin from binding to ActRIIB, e.g., via an ActRII antibody, the formation of skeletal muscle can be induced. Regulation of the activin receptor II A (ActRIIA) also plays a role in the regulation of muscle growth (Morvan et al. 2017).

Exemplary ActRII antibodies that bind to ActRIIA and ActRIIB were shown to not only increase lean muscle mass, but also to decrease fat mass and improve glycemic control in human clinical studies (WO2010125003A1, WO2018116201A1, the contents of which are incorporated in their entirety, and Heymsfield et al. 2021; 4(1):e2033457, JAMA). Exemplary ActRII antibodies that may be used in some embodiments include the human recombinant antibodies, isolated and structurally characterized, as described in WO2010125003A1.

In some embodiments, exemplary antibodies of the disclosure include the sequences of bimagrumab (BYM338). The below table provides the relevant CDR, VH, VL, HC, and LC amino acid sequences for bimagrumab.

Bimagrumab Sequences
Bimagrumab Heavy Chain Complementarity Determining Regions (CDRs)
CDRH1-GYTFTSSYIN (SEQ ID NO: 1)
CDRH2-TINPVSGSTSYAQKFQ (SEQ ID NO: 2)
CDRH3-GGWFDY (SEQ ID NO: 3)
Bimagrumab Light Chain Complementarity Determining Regions (CDRs)
CDRL1-TGTSSDVGSYNYVN(SEQ ID NO: 4)
CDRL2-MIYGVSKRPS (SEQ ID NO: 5)
CDRL3-GTFAGGSYYG (SEQ ID NO: 6)
Bimagrumab Variable Heavy Chain (VH)
QVQLVQSGAEVKKPGASVKVSCKASGYTFTSSYINWVRQAPGQGLEWMGTINPVSGSTSY
AQKFQGRVTMTRDTSISTAYMELSRLRSDDTAVYYCARGGWFDYWGQGTLVTVSS
(SEQ ID NO: 7)
Bimagrumab Variable Light Chain (VL)
QSALTQPASVSGSPGQSITISCTGTSSDVGSYNYVNWYQQHPGKAPKLMIYGVSKRPSGV
SNRFSGSKSGNTASLTISGLQAEDEADYYCGTFAGGSYYGVFGGGTKLTVLGQ
(SEQ ID NO: 8)
Bimagrumab Heavy Chain (HC)
QVQLVQSGAEVKKPGASVKVSCKASGYTFTSSYINWVRQAPGQGLEWMGTINPVSGSTSY
AQKFQGRVTMTRDTSISTAYMELSRLRSDDTAVYYCARGGWFDYWGQGTLVTVSSASTKG
PSVFPLAPSSKSTSGGTAALGCLVKDYFPEPVTVSWNSGALTSGVHTFPAVLQSSGLYSL
SSVVTVPSSSLGTQTYICNVNHKPSNTKVDKRVEPKSCDKTHTCPPCPAPEAAGGPSVFL
FPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLTVDKSRWQQGNV
FSCSVMHEALHNHYTQKSLSLSPGK
(SEQ ID NO: 9)
Bimagrumab Light Chain (LC)
QSALTQPASVSGSPGQSITISCTGTSSDVGSYNYVNWYQQHPGKAPKLMIYGVSKRPSGV
SNRFSGSKSGNTASLTISGLQAEDEADYYCGTFAGGSYYGVFGGGTKLTVLGQPKAAPSV
TLFPPSSEELQANKATLVCLISDFYPGAVTVAWKADSSPVKAGVETTTPSKQSNNKYAAS
SYLSLTPEQWKSHRSYSCQVTHEGSTVEKTVAPTECS (SEQ ID NO: 10)

In some embodiments, exemplary ActRII antibodies of the disclosure comprise a variable heavy chain comprising the CDR amino acid sequences of SEQ ID NO: 1 (CDRH1), SEQ ID NO: 2 (CDRH2), and SEQ ID NO: 3 (CDRH3).

In some embodiments, exemplary ActRII antibodies of the disclosure comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII antibodies of the disclosure comprise a variable heavy chain comprising the CDR amino acid sequences of SEQ ID NO: 1 (CDRH1), SEQ ID NO: 2 (CDRH2), and SEQ ID NO: 3 (CDRH3); and comprise a variable light chain comprising the CDR amino acid sequences of SEQ ID NO: 4 (CDRL1), SEQ ID NO: 5 (CDRL2), and SEQ ID NO: 6 (CDRL3).

In some embodiments, exemplary ActRII antibodies of the disclosure comprise a variable heavy chain comprising the amino acid sequence of SEQ ID NO: 7, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and a variable light chain comprising the amino acid sequence of SEQ ID NO: 8, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, exemplary ActRII antibodies of the disclosure comprise a heavy chain comprising the amino acid sequence of SEQ ID NO: 9, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto; and a light chain comprising the amino acid sequence of SEQ ID NO: 10, or an amino acid sequence of at least 80%, 81%, 82%, 83%, 84%, 85%, 86%, 87%, 88%, 89%, 90%, 91%, 92%, 93%, 94%, 95%, 96%, 97%, 98%, or 99% sequence identity thereto.

In some embodiments, an ActRII antibody binds to ActRIIB with a KD of 100 nM or less, 10 nM or less, 1 nM or less. In exemplary embodiments, an ActRII antibody binds to ActRIIB with an affinity of 100 pM or less (i.e. 100 pM, 50 pM, 10 pM, 1 pM or less). In some embodiments, an ActRII antibody binds to ActRIIB with an affinity of between 10 and 20 pM.

In some embodiments, an ActRII antibody binds to ActRIIB with a 5-fold, 10-fold, 50-fold, even 100-fold greater affinity than to ActRIIA. In some embodiments, an ActRII antibody binds to ActRIIA with an affinity of 100 pM or more (i.e. 250 pM, 500 pM, 1 nM, 5 nM or more).

### Other ActRII Pathway Agents

In other embodiments, the treatments described herein include an agent that binds an ActRII receptor ligand, e.g., binds myostatin and/or activin directly. Such agents include, but are not limited to, a myostatin inhibitor (e.g., a myostatin antibody, or a myostatin small molecule antagonist), an activin inhibitor (e.g., an activin antibody, or an activin small molecule antagonist), or a soluble extracellular portion of ActRIIB or ActRIIA that can act as a “ligand sink” optionally further stabilized with an Fc. In some embodiments, the treatments described herein include bi-specific antibodies that can bind both activin and myostatin. Accordingly, in some embodiments, provided herein are methods for treating a metabolic disorder of the disclosure, comprising administering to a subject in need an ActrII pathway agent (other than an ActRII antibody), wherein the agent is selected from the group consisting of a myostatin inhibitor, an activin inhibitor, a soluble portion of an ActRII receptor, or a bi-specific antibody.

### III. ActRII Antibody Treatments

An ActRII antibody (or other ActRII pathway agent) may be administered to a subject in need thereof to treat a metabolic disorder. An ActRII antibody treatment of the disclosure may include an ActRII antibody dosage regimen. In some embodiments, an ActRII antibody treatment may include one or more optional loading doses of an ActRII antibody (or other ActRII pathway agent) in addition to a dosage regimen. In exemplary embodiments, an ActRII antibody comprising the amino acid sequence of SEQ ID NOS: 1-6 and/or SEQ ID NOS: 7 and 8 (or a sequence with sequence identity of at least 90% thereto) is administered in a dosage regimen. In further exemplary embodiments, an ActRII antibody comprising the sequence of SEQ ID NOS: 1-6 and/or SEQ ID NOS: 7 and 8 (or a sequence with sequence identity of at least 90% thereto) is administered in a loading dose and a dosage regimen.

In some embodiments, an ActRII antibody treatment as provided herein comprises a dosage regimen, comprising administration of an ActRII antibody to a subject in need thereof about once every 8 weeks to about once every 16 weeks, e.g., about once every 12 weeks, in a dose of about 3-50 mg/kg, e.g., about 30 mg/kg, intravenously. In some embodiments, a dosage regiment comprises administration of an ActRII antibody in a dose of about 30 mg/kg intravenously, to a subject in need thereof about every 12 weeks.

In other embodiments, of the ActRII antibody treatments contemplated herein, an ActRII antibody is administered in a dose of about 200 mg to about 400 mg to a subject in need thereof.

As provided herein, an ActRII antibody treatment may be administered by any route, for example in exemplary embodiments, an ActRII antibody may be administered intravenously or subcutaneously.

### Loading Dose

In some embodiments, the administration of a loading dose in addition to a dosage regimen may improve the outcome of an ActRII antibody treatment. Without being bound by theory, it is thought that administration of a loading dose may maintain the level of the ActRII antibody in the serum of the subject over time. Accordingly, in some embodiments, a loading dose is provided herein.

In some embodiments, a loading dose maintains the serum concentration of the ActRII antibody at or above the saturation concentration of the ActRII receptor. In some embodiments, about 10 pg/ml of an ActRII antibody is a sufficient concentration to saturate ActRII receptor signaling in a subject.

In some embodiments, the “C_{trough}” refers to the lowest serum concentration of an ActRII antibody during the subject's treatment, most often the serum level of an ActRII antibody prior to the next dose. In some embodiments, the C_{trough} is about the same concentration as the ActRII receptor saturation concentration. In some embodiments, the C_{trough} is about 3 μg/ml to about 30 μg/ml. In some embodiments, the C_{trough} is about 10 μg/ml. In some embodiments, a loading dose maintains the C_{trough} at about 3 to about 30 μg/ml. In some embodiments, a loading dose maintains the C_{trough} at about 4 μg/ml; at about 5 μg/ml; at about 6 μg/ml; at about 7 μg/ml; at about 8 μg/ml; at about 9 μg/ml; at about 10 μg/ml; at about 11 μg/ml; at about 12 μg/ml; at about 13 μg/ml; at about 14 μg/ml; at about 15 μg/ml; at about 16 μg/ml; at about 17 μg/ml; at about 18 μg/ml; at about 19 μg/ml; at about 20 μg/ml; at about 21 μg/ml; at about 22 μg/ml; at about 23 μg/ml; at about 24 μg/ml; at about 25 μg/ml; at about 26 μg/ml; at about 27 μg/ml; at about 28 μg/ml; at about 29 μg/ml; or at about 30 μg/ml in the serum of the subject. In some embodiments, a desired C_{trough} is selected to measure the efficiency of the bimagrumab treatment. In some embodiments, a loading dose maintains the C_{trough} at about 10 μg/ml during the course of the treatment.

In some embodiments, a loading dose lowers the dose required in the dosage regimen to achieve a desired C_{trough} relative to the dosage regimen without the loading dose. Accordingly, in some embodiments, a loading dose is thought to maximize the efficacy and safety of the ActRII antibody treatment, and is included in the treatment.

In some embodiments, one or more loading doses of an ActRII antibody are administered to a subject in need thereof at day 0 or week 0 prior to the start of a subsequent dosage regimen. In some embodiments, one or more loading dose of an ActRII antibody are provided 1 day, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, about 11 weeks, or about 12 weeks before a ActRII antibody dosage regimen is administered. In exemplary embodiments, a loading dose is administered about 4 weeks before the start of a dosage regimen.

In some embodiments, a second administration of one or more loading doses are given after about 6 months, about 8 months, about 10 months, about 12 months, about 14 months, about 16 months, about 18 months, about 20 months, or about 24 months of administration of the dosage regimen. In some embodiments, a subject is administered one or more loading doses followed by a dosage regimen, and is then administered a second round of treatment, e.g. a second demonstration of one or more loading doses, followed by a repeated dosing regimen. Likewise the treatment could include yet a third set of one or more loading doses, followed by a third round of a dosing regimen, and so on and so forth. In some embodiments, one or more loading doses are administered periodically or on a regular basis in addition to the dosage regimen. In exemplary embodiments, a loading dose is administered first about 4 weeks prior to the start of the dosage regimen, and administered about yearly thereafter in addition to the dosage regimen.

In some embodiments, a loading dose is administered prior to an ActRII antibody dosage regimen, wherein the ActRII antibody dosage regimen comprises administration of the ActRII antibody about once every 8 weeks to about once every 16 weeks, e.g., about once every 12 weeks, in a dose of about 3-50 mg/kg, e.g., about 30 mg/kg, wherein the dose is administered intravenously.

In some embodiments, a loading dose is a dose of an ActRII antibody at the same concentration, a lower concentration, or a higher concentration as the dose of and ActRII antibody administered in the dosage regimen. Accordingly, a loading dose may be about 3 mg/kg per the subject's body weight to about 50 mg/kg per the subject's body weight, and all amounts in between. In exemplary embodiments, a loading dose between about 3 mg/kg per the subject's body weight to about 50 mg/kg per the subject's body weight is administered intravenously. In some exemplary embodiments, a loading dose of an ActRII antibody of about 30 mg/kg is administered to a subject about 4 weeks before the start of a dosage regimen, and is administered intravenously.

In some embodiments, a loading dose of an ActRII antibody is administered to a subject in need thereof, for example intravenously, in a dose of about 3 mg/kg; in some embodiments in a dose of about 4 mg/kg; in some embodiments in a dose of about 5 mg/kg; in some embodiments in a dose of about 6 mg/kg; in some embodiments in a dose of about 7 mg/kg; in some embodiments in a dose of about 8 mg/kg; in some embodiments in a dose of about 9 mg/kg; in some embodiments in a dose of about 10 mg/kg; in some embodiments in a dose of about 11 mg/kg; in some embodiments in a dose of about 12 mg/kg; in some embodiments in a dose of about 13 mg/kg; in some embodiments in a dose of about 14 mg/kg; in some embodiments in a dose of about 15 mg/kg; in some embodiments in a dose of about 16 mg/kg; in some embodiments in a dose of about 17 mg/kg; in some embodiments in a dose of about 18 mg/kg; in some embodiments in a dose of about 19 mg/kg; in some embodiments in a dose of about 20 mg/kg; in some embodiments in a dose of about 21 mg/kg; in some embodiments in a dose of about 22 mg/kg; in some embodiments in a dose of about 23 mg/kg; in some embodiments in a dose of about 24 mg/kg; in some embodiments in a dose of about 25 mg/kg; in some embodiments in a dose of about 26 mg/kg; in some embodiments in a dose of about 27 mg/kg; in some embodiments in a dose of about 28 mg/kg; in some embodiments in a dose of about 29 mg/kg; in some embodiments in a dose of about 30 mg/kg; in some embodiments in a dose of about 31 mg/kg; in some embodiments in a dose of about 32 mg/kg; in some embodiments in a dose of about 33 mg/kg; in some embodiments in a dose of about 34 mg/kg; in some embodiments in a dose of about 35 mg/kg; in some embodiments in a dose of about 36 mg/kg; in some embodiments in a dose of about 37 mg/kg; in some embodiments in a dose of about 38 mg/kg; in some embodiments in a dose of about 39 mg/kg; in some embodiments, in a dose of about 40 mg/kg; in some embodiments in a dose of about 41 mg/kg; in some embodiments in a dose of about 42 mg/kg; in some embodiments in a dose of about 43 mg/kg; in some embodiments in a dose of about 44 mg/kg; in some embodiment in a dose of about 45 mg/kg; in some embodiments in a dose of about 46 mg/kg; in some embodiments in a dose of about 47 mg/kg; in some embodiments in a dose of about 48 mg/kg; in some embodiments in a dose of about 49 mg/kg; and in some embodiments in a dose of about 50 mg/kg. In exemplary embodiments, an ActRII antibody of a loading dose is about 10 mg/kg per the subject's body weight. In other exemplary embodiments, an ActRII antibody of a loading dose is about 30 mg/kg per the subject's body weight and is administered intravenously.

In some exemplary embodiments, a loading dose of about 3-50 mg/kg is administered intravenously about 0 to about 12 weeks prior to the start of an ActRII antibody dosage regimen, wherein the dosage regimen comprises administration of an ActrII antibody about once every 8 weeks to about once every 16 weeks, in a dose of about 3 mg/kg to about 50 mg/kg (e.g. about 3 mg/kg, about 10 mg/kg, about 30 mg/kg, or about 50 mg/kg), wherein the ActRII antibody dosage regimen is administered intravenously. In some exemplary embodiments, a loading dose of about 30 mg/kg is administered intravenously about 4 weeks prior to the start of an ActRII antibody dosage regimen, wherein the dosage regimen comprises administration of an ActrII antibody about once every 12 weeks, in a dose of about 30 mg/kg, wherein the ActRII antibody dosage regimen is administered intravenously.

In other exemplary embodiments, a loading dose of an ActRII antibody may be about 200 mg to about 400 mg per dose, and not determined by a subject's body weight, or fixed at a dose applicable across a range of subject body weights, and may be administered subcutaneously. In some exemplary embodiments, a loading dose of an ActRII antibody may be about 200 mg to about 400 mg per dose and is administered subcutaneously. In some embodiments, a loading dose of an ActRII antibody is about 200 mg per dose. In some embodiments, a loading dose of an ActRII antibody is about 250 mg per dose. In some embodiments, a loading dose of an ActRII antibody is about 300 mg per dose. In some embodiments, a loading dose of an ActRII antibody is about 350 mg per dose. In some embodiments, a loading dose of an ActRII antibody is about 400 mg per dose. In some embodiments, a loading dose of an ActRII antibody is about 300 mg per dose and is administered subcutaneously.

### Dosage Regimen

A dosage regimen as contemplated herein is a regular, repeated dosage of an ActRII antibody (or other ActRII pathway agent).

In some exemplary embodiments, a dosage regimen may be administered to a subject in need thereof in a dose of about 3 mg/kg to about 50 mg/kg. In some exemplary embodiments, a dosage regimen may be administered to a subject in need thereof in a dose of about 3 mg/kg to about 50 mg/kg intravenously.

In some embodiments a dosage regimen may be administered to a subject in need thereof in a dose of about 3 mg/kg; in some embodiments in a dose of about 4 mg/kg; in some embodiments in a dose of about 5 mg/kg; in some embodiments in a dose of about 6 mg/kg; in some embodiments in a dose of about 7 mg/kg; in some embodiments in a dose of about 8 mg/kg; in some embodiments in a dose of about 9 mg/kg; in some embodiments in a dose of about 10 mg/kg; in some embodiments in a dose of about 11 mg/kg; in some embodiments in a dose of about 12 mg/kg; in some embodiments in a dose of about 13 mg/kg; in some embodiments in a dose of about 14 mg/kg; in some embodiments in a dose of about 15 mg/kg; in some embodiments in a dose of about 16 mg/kg; in some embodiments in a dose of about 17 mg/kg; in some embodiments in a dose of about 18 mg/kg; in some embodiments in a dose of about 19 mg/kg; in some embodiments in a dose of about 20 mg/kg; in some embodiments in a dose of about 21 mg/kg; in some embodiments in a dose of about 22 mg/kg; in some embodiments in a dose of about 23 mg/kg; in some embodiments in a dose of about 24 mg/kg; in some embodiments in a dose of about 25 mg/kg; in some embodiments in a dose of about 26 mg/kg; in some embodiments in a dose of about 27 mg/kg; in some embodiments in a dose of about 28 mg/kg; in some embodiments in a dose of about 29 mg/kg; in some embodiments in a dose of about 30 mg/kg; in some embodiments in a dose of about 31 mg/kg; in some embodiments in a dose of about 32 mg/kg; in some embodiments in a dose of about 33 mg/kg; in some embodiments in a dose of about 34 mg/kg; in some embodiments in a dose of about 35 mg/kg; in some embodiments in a dose of about 36 mg/kg; in some embodiments in a dose of about 37 mg/kg; in some embodiments in a dose of about 38 mg/kg; in some embodiments in a dose of about 39 mg/kg; in some embodiments, in a dose of about 40 mg/kg; in some embodiments in a dose of about 41 mg/kg; in some embodiments in a dose of about 42 mg/kg; in some embodiments in a dose of about 43 mg/kg; in some embodiments in a dose of about 44 mg/kg; in some embodiment in a dose of about 45 mg/kg; in some embodiments in a dose of about 46 mg/kg; in some embodiments in a dose of about 47 mg/kg; in some embodiments in a dose of about 48 mg/kg; in some embodiments in a dose of about 49 mg/kg; and in some embodiments in a dose of about 50 mg/kg. In exemplary embodiments, an ActRII antibody of a dosage regimen is administered to a subject in need thereof in a dose of 10 mg/kg. In other exemplary embodiments an ActRII antibody of a dosage regimen is administered to a subject in need thereof in a dose of 30 mg/kg.

In some embodiments an ActRII antibody is administered to a subject in need thereof two, three, four, or five or more times in a dosage regimen. In some embodiments, an ActRII antibody in a dosage regimen is administered about every 8 weeks, about every 9 weeks, about every 10 weeks, about every 11 weeks, about every 12 weeks, about every 13 weeks, about every 14 weeks, about every 15 weeks, or about every 16 weeks. In some embodiments, an ActRII antibody is administered every quarter. In some embodiments, an ActRII antibody of a dosage regimen is administered in a dose of about 3 mg/kg to about 50 mg/kg, about every 8 to about 16 weeks, intravenously. In some embodiments, an ActRII antibody of a dosage regimen is administered in a dose of about 30 mg/kg about every 12 weeks, intravenously.

In some embodiments, an ActRII antibody of a dosage regimen is administered intravenously in a dose of about 30 mg/kg about every 12 weeks, optionally following the administration of a loading dose.

In some embodiments, an ActRII antibody of a dosage regimen is administered intravenously in a dose of about 3 to about 50 mg/kg about every 12 weeks, optionally following the administration of a loading dose, optionally wherein the loading dose is administered about 4 weeks prior to the start of the dosage regimen, and optionally, about every year thereafter of the dosage regimen. In some embodiments, an ActRII antibody of a dosage regimen is administered in a dose of about 30 mg/kg about every 12 weeks, optionally following the administration of a loading dose, optionally wherein the loading dose is administered about 4 weeks prior to the start of the dosage regimen and, optionally about every year thereafter of the dosage regimen.

In some exemplary embodiments, a loading dose of about 3-50 mg/kg is administered intravenously about 0 to about 12 weeks prior to the start of an ActRII antibody dosage regimen, wherein the dosage regimen comprises administration of an ActrII antibody about once every 8 weeks to about once every 16 weeks, in a dose of about 3 mg/kg to about 50 mg/kg, wherein the ActRII antibody dosage regimen is administered intravenously. In some exemplary embodiments, a loading dose of about 30 mg/kg is administered intravenously about 4 weeks prior to the start of an ActRII antibody dosage regimen, wherein the dosage regimen comprises administration of an ActrII antibody about once every 12 weeks, in a dose of about 30 mg/kg, wherein the ActRII antibody dosage regimen is administered intravenously.

In some embodiments an ActRII antibody of a dosage regimen is administered in a dosage regimen of about 100 to about 600 mg, once, twice, three, or more times weekly. In some embodiments an ActRII antibody is administered in a dosage regimen of about 200 to about 400 mg, for example, about 300 mg, once, twice, three, or more times weekly, or once every 2 weeks. In some embodiments, an ActRII antibody is administered in a dosage regimen of about 300 mg once weekly. In some embodiments, an ActRII antibody is administered in a dosage regimen of about 300 mg once weekly subcutaneously.

In other exemplary embodiments, an ActRII antibody of a dosage regimen is administered in a weight of about 200 mg to about 400 mg per dose. In some exemplary embodiments, an ActRII antibody of a dosage regimen is administered in a weight of about 200 mg to about 400 mg per dose subcutaneously. In some exemplary embodiments, an ActRII antibody of a dosage regimen is administered in a weight of about 200 mg. In some exemplary embodiments, an ActRII antibody of a dosage regimen is administered in a weight of about 250 mg. In some exemplary embodiments, an ActRII antibody of a dosage regimen is administered in a weight of about 300 mg. In some exemplary embodiments, an ActRII antibody of a dosage regimen is administered in a weight of about 350 mg. In some exemplary embodiments, an ActRII antibody of a dosage regimen is administered in a weight of about 400 mg. In some exemplary embodiments, an ActRII antibody of a dosage regimen is administered in a weight of about 300 mg subcutaneously.

In some embodiments, an ActRII antibody of a dosage regimen is administered in a dose of about 200 mg. In some embodiments, an ActRII antibody of a dosage regimen is administered in a dose of about 200 mg once weekly. In some embodiments, an ActRII antibody of a dosage regimen is administered in a dose of about 200 mg twice weekly. In some embodiments an ActRII antibody of a dosage regimen is administered in a dose of 200 mg thrice weekly. In some embodiments, an ActRII antibody of a dosage regimen is administered in a dose of about 200 mg every 2 weeks. In some embodiments, an ActRII antibody of a dosage regimen is administered in a dose of about 200 mg subcutaneously.

In exemplary embodiments, an ActRII antibody of a dosage regimen is administered in a dose of about 300 mg. In exemplary embodiments, an ActRII antibody of a dosage regimen is administered in a dose of about 300 mg once weekly. In some embodiments, an ActRII antibody of a dosage regimen is administered in a dose of about 300 mg twice weekly. In some embodiments an ActRII antibody of a dosage regimen is administered in a dose of 300 mg thrice weekly. In some embodiments, an ActRII antibody of a dosage regimen is administered in a dose of about 300 mg every 2 weeks. In exemplary embodiments, an ActRII antibody of a dosage regimen is administered in a dose of about 300 mg subcutaneously. In exemplary embodiments, an ActRII antibody of a dosage regimen antibody is administered in a dose of about 300 mg once weekly subcutaneously.

In some embodiments, an ActRII antibody of a dosage regimen is administered in a dose of about 400 mg. In some embodiments, an ActRII antibody of a dosage regimen is administered in a dose of about 400 mg once weekly. In some embodiments, an ActRII antibody of a dosage regimen is administered in a dose of about 400 mg twice weekly. In some embodiments an ActRII antibody of a dosage regimen is administered in a dose of 400 mg thrice weekly. In some embodiments, an ActRII antibody of a dosage regimen is administered in a dose of about 400 mg every 2 weeks. In some embodiments, an ActRII antibody of a dosage regimen is administered in a dose of about 400 mg once weekly subcutaneously.

### Routes of Administration

In some embodiments, an ActRII antibody of a loading dose and/or an ActRII antibody of a dosage regimen may be delivered orally, intravenously, intranasally, transdermally, intraperitoneally, intramuscularly, intrapulmonary, vaginally, rectally, or intraocularly. In exemplary embodiments an ActRII antibody of a loading dose and/or an ActRII antibody of a dosage regimen are administered intravenously. In other exemplary embodiments an ActRII antibody of a loading dose and/or an ActRII antibody of a dosage regimen are administered subcutaneously.

In some exemplary embodiments, an ActrII antibody is administered about once every 8 weeks to about once every 16 weeks, in a dose of about 3 mg/kg to about 50 mg/kg, intravenously.

In other exemplary embodiments, a loading dose of about 3 to about 50 mg/kg is administered intravenously about 0 to about 12 weeks prior to the start of an ActRII antibody dosage regimen, wherein the dosage regimen comprises administration of an ActrII antibody about once every 8 weeks to about once every 16 weeks, in a dose of about 3 mg/kg to about 50 mg/kg, and wherein the ActRII antibody dosage regimen is administered intravenously. In some exemplary embodiments, a loading dose of about 30 mg/kg is administered intravenously about 4 weeks prior to the start of an ActRII antibody dosage regimen, wherein the dosage regimen comprises administration of an ActrII antibody about once every 12 weeks, in a dose of about 30 mg/kg, and wherein the ActRII antibody dosage regimen is administered intravenously.

### Subjects and Treatment

As put forth herein, ActRII treatments are useful for the treatment of metabolic disorders in a subject in need thereof, including but not limited to obesity, diabetes (Type I and II), metabolic syndrome, anti-psychotic drug-associated obesity, glucocorticoid-induced obesity, hypothalamic obesity associated with craniopharyngioma, and complex and monogenetic disorders associated with obesity. The monogenetic disorders associated with obesity in humans, may include but are not limited to Bardet-Biedl syndrome, and a disorder arising from a mutation in one or more of the following genes ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B, or combinations thereof. A metabolic disorder may also be associated with a complex genetic disorder, e.g., Prader-Willi syndrome.

In some embodiments, an ActRII antibody treatment treats an obesity related disorder or condition. The obesity related disorder or condition may include but is not limited to one or more of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

In some embodiments, reference to cardiometabolic disease includes cardiometabolic syndrome, which includes, but is not limited to, insulin resistance, impaired glucose tolerance, dyslipidemia, hypertension, and central adiposity. In some embodiments, cardiometabolic syndrome includes coronary heart disease and risk for or previous experience of ischemic stroke.

In exemplary embodiments, the metabolic disorder is treated with an ActRII antibody dosage regimen as described herein, and optionally, a loading dose of an ActRII antibody. An ActRII antibody treatment as described herein may comprise a dosage regimen and/or loading dose of an ActRII antibody comprising the sequence of SEQ ID NOS: 1-6 and/or SEQ ID NOS: 7 and 8, or a sequence with sequence identity of at least 90% thereto, and in some embodiments be administered every 12 weeks at a dosage of 10 μg/ml. In other embodiments, an ActRII antibody treatment as described herein may comprise a dosage regimen and/or loading dose of an ActRII antibody comprising the sequence of SEQ ID NOS: 1-6 and/or SEQ ID NOS: 7 and 8, or a sequence with sequence identity of at least 90% thereto, and in some embodiments be administered about every 12 weeks at a dosage of about 30 μg/ml intravenously. In other embodiments, an ActRII antibody treatment as described herein may comprise a dosage regimen and/or loading dose of an ActRII antibody comprising the sequence of SEQ ID NOS: 1-6 and/or SEQ ID NOS: 7 and 8, or a sequence with sequence identity of at least 90% thereto, and in some embodiments be administered every week weeks at a dosage of 300 mg per dose, e.g. subcutaneously. In some embodiments, one or more loading doses of an ActRII antibody comprising the sequence of SEQ ID NOS: 1-6 and/or SEQ ID NOS: 7 and 8, or a sequence with sequence identity of at least 90% thereto is administered 4 weeks prior to the start of the dosage regimen, and optionally, yearly thereafter.

In some embodiments, a subject in need of the treatments described herein is a subject who is overweight. In some embodiments, a subject in need of the treatments described herein includes a subject with a BMI of 30 or greater. In some embodiments, a subject in need of the treatments described herein includes a subject with a BMI of 27 or greater with an obesity related co-morbidity.

In some embodiments, a subject in need of the treatments described herein lacks glycemic control.

Subjects can be of any age, including youth. In some embodiments, the subject is over 40 years old, over 45 years old, over 50 years old, over 60 years old, or over 80 years old. In exemplary embodiments the subject is 45 or older.

In some embodiments, an ActRII antibody treatment reduces fat mass in the subject. In some embodiments, the treatment reduces fat mass in a subject by at least 5% (e.g., 5%-30%) over the treatment period.

In some embodiments, an ActRII antibody treatment increases lean mass in the subject. In some embodiments, the treatment increases lean mass in a subject by at least 1% (e.g., 1%-10%) over the treatment period.

In some embodiments, an ActRII antibody treatment reduces fat mass and increases lean mass in the subject. In some embodiments, the treatment reduces fat mass in a subject by at least 5% (e.g., 5%-30%) and increases lean mass by at least 1% (e.g., 1%-10%) over the treatment period.

In some embodiments, an ActRII antibody treatment reduces fat mass and maintains lean mass in the subject. In some embodiments, the treatment reduces fat mass in a subject by at least 5% (e.g., 5%-30%) and maintains lean mass over the treatment period.

In some embodiments, an ActRII antibody treatment reduces body weight in the subject. In some embodiments, the treatment reduces body weight in a subject by at least 5%, at least 10%, at least 20%, at least 30%, at least 40%, or at least 50% over the treatment period.

In some embodiments, an ActRII antibody treatment reduces central adiposity in the subject. In some embodiments, the treatment reduces central adiposity in a subject by at least 2% (e.g., 2%-20%) over the treatment period.

In some embodiments an ActRII antibody treatment reduces liver fat and/or non-liver visceral fat in the subject. In some embodiments, the treatment reduces central adiposity in a subject by at least 2% (e.g., 2%-20%) over the treatment period as assessed by magnetic resonance imaging (MRI).

In some embodiments, fat mass in response to an ActRII antibody treatment is measured with bioelectrical impedance analysis (BIA), dual X-ray absorptiometry (DXA), magnetic resonance imaging (MRI), and/or waist circumference.

In some embodiments, an ActRII antibody treatment improves glycemic control in the subject.

In some embodiments, glycemic control in response to an ActRII antibody treatment is measured by glucose and insulin levels, and the HOMA2 model is applied (www.dtu.ox.ac.uk/homacalculator/).

In some embodiments, the efficacy of an ActRII antibody treatment is determined by about 100%, about 90%, about 80%, about 75%, about 50%, or about 25% improvement in at least one of the following measurements: body weight; bioelectrical impedance analysis (BIA) of lean mass and/or fat mass; waist to hip ratio; waist to height ratio; dual X-ray absorptiometry (DXA) of lean mass and/or fat mass; magnetic resonance imaging (MRI) of lean mass and/or fat mass; waist circumference; decreased BMI; blood lipids profile; leptin, lectin, adiponectin, and adipsin levels; IL-8 and/or IL-6 levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; the homeostasis model assessment 2 (HOMA2); and physical activity monitoring via actigraphy.

In some embodiments, the “C_{trough}” in a sample from a subject in treatment may be measured to determine the efficacy of the treatment. In some embodiments, a treatment is efficacious when the C_{trough} of the sample from the subject is at least about 500%, about 400%, about 300%, about 200%, about 100%, about 90%, about 80%, or about 75% of a desired “C_{trough}”. In some embodiments, the desired “C_{trough}” is about 3 μg/ml to about 30 μg/ml. In some embodiments, the desired “C_{trough}” is about 10 μg/ml.

### IV. Pharmaceutical Compositions

In some embodiments, an ActRII antibody is in a pharmaceutical composition. In exemplary embodiments, the composition includes an ActRII antibody comprising the sequence of SEQ ID NOS: 1-6 and/or SEQ ID NOS: 7 and 8 (or a sequence with sequence identity of at least 90% thereto). In some embodiments the pharmaceutical composition is formulated for intravenous administration. In some embodiments the pharmaceutical composition is formulated for subcutaneous administration.

In some embodiments the composition includes an excipient, or carrier, e.g., an aqueous carrier. A variety of aqueous carriers can be used, e.g., buffered saline. The compositions may contain pharmaceutically acceptable auxiliary substances as those required to approximate physiological conditions such as pH and buffering agents, toxicity countering agents, e.g., disodium phosphate dihydrate, monosodium phosphate, sodium acetate, sodium chloride, potassium chloride, calcium chloride, hydrochloric acid, sodium hydroxide, L-histidine, L-histidine hydrochloride, and sodium lactate. The concentration of active agent in these formulations can vary and are selected based on fluid volumes, viscosities, and body weight in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)). The compositions may contain pharmaceutically acceptable auxiliary substances such as those that contribute to the stability and activity of the pharmacologically active agent or agents, including but not limited to trehalose, sucrose, or other sugars and polysorbate 20, polysorbate 60, polysorbate 80 or other emulsifiers or stabilizers.

### Exemplary Embodiments

### Embodiments Set I

Embodiment I-1. A method of treating a metabolic disorder in a subject in need thereof comprising administering to the subject an ActRII antibody loading dose followed by an ActRII antibody dosage regimen.

Embodiment I-2. A method of treating a metabolic disorder in a subject in need thereof, wherein the method comprises an ActRII antibody dosage regimen, and wherein an ActRII antibody is administered to the subject about every 12 weeks.

Embodiment I-3. The method of embodiment I-2, wherein the ActRII antibody dosage regimen follows the administration of an ActRII antibody loading dose.

Embodiment I-4. The method of embodiment I-1 or I-3, wherein the ActRII antibody loading dose is about 3 mg/kg to about 50 mg/kg.

Embodiment I-5. The method of embodiment I-1 or I-3, wherein the AcRII antibody loading dose is about 10 mg/kg.

Embodiment I-6. The method of embodiment I-1 or I-3, wherein the AcRII antibody loading dose is about 30 mg/kg.

Embodiment I-7. The method of any one of embodiments I-1 to I-6, wherein the ActRII antibody dosage regimen starts about 1 day, about 2 days, about 3 days, about 4 days, about 5 days, about 6 days, about 1 week, about 2 weeks, about 3 weeks, about 4 weeks, or about 5 weeks after the loading dose of the ActRII antibody.

Embodiment I-8. The method of any one of embodiments I-1 to I-7, wherein the ActRII antibody dosage regimen comprises administration of an ActRII antibody in a dose of about 3 mg/kg to about 50 mg/kg.

Embodiment I-9. The method of any one of embodiments I-1 to I-7, wherein the ActRII antibody dosage regimen comprises administration of an ActRII antibody in a dose of about 10 mg/kg.

Embodiment I-10. The method of any one of embodiments I-1 to I-7, wherein the ActRII antibody dosage regimen comprises administration of an ActRII antibody in a dose of about 30 mg/kg.

Embodiment I-11. The method of any one of embodiments I-1 to I-10, wherein the ActRII antibody dosage regimen comprises administration of an ActRII antibody about every 4 weeks, about every 8 weeks, about every 12 weeks, or about every 16 weeks.

Embodiment I-12. The method of embodiment I-11, wherein the ActRII antibody dosage regimen comprises administration of an ActRII antibody about every 12 weeks.

Embodiment I-13. The method of any one of embodiments I-1 or I-3 to I-12, wherein the ActRII antibody loading dose is administered intravenously.

Embodiment I-14. The method of any one of embodiments I-1 to I-12, wherein the ActRII antibody dosage regimen is administered intravenously.

Embodiment I-15. The method of any one of embodiments I-1 or I-3 to I-12, wherein the ActRII antibody loading dose and the ActRII antibody dosage regimen are administered intravenously.

Embodiment I-16. The method of any one of embodiments I-1 or I-3 to I-12, wherein the ActRII antibody of the loading dose comprises the amino acid sequence of SEQ ID NOS: 1-6

Embodiment I-17. The method of any one of embodiments I-1 or I-3 to I-12, wherein the ActRII antibody of the loading dose comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto.

Embodiment I-18. The method of any one of embodiments I-1 or I-3 to I-12 wherein the loading dose of the ActRII antibody comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

Embodiment I-19. The method of anyone embodiments I-1 or I-3 to I-12, wherein the ActRII antibody of the loading dose is specific for ActRIIA and ActRIIB.

Embodiment I-20. The method of any one of embodiments I-1 to I-19, wherein the ActRII antibody dosage regimen comprises administration of an ActRII antibody comprising the amino acid sequence of SEQ ID NOS: 1-6.

Embodiment I-21. The method of any one of embodiments I-1 to I-19, wherein the ActRII antibody dosage regimen comprises administration of an ActRII antibody comprising: the amino acid sequence of SEQ ID NO: 7, or a sequence with sequence identity of at least 90% thereto, and comprising the amino acid sequence of SEQ ID NO: 8, or a sequence with sequence identity of at least 90% thereto.

Embodiment I-22. The method of any one of embodiments I-1 to I-19, wherein the ActRII antibody dosage regimen comprises administration of an ActRII antibody comprising: the amino acid sequence of SEQ ID NO: 9, or a sequence with sequence identity of at least 90% thereto, and comprising the amino acid sequence of SEQ ID NO: 10, or a sequence with sequence identity of at least 90% thereto.

Embodiment I-23. The method of any one of embodiments I-1 to I-19, wherein the ActRII antibody dosage regimen comprises administration of an ActRII antibody specific for ActRIIA and ActRIIB.

Embodiment I-24. The method of any one of embodiments I-1 to I-23, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

Embodiment I-25. The method of embodiment I-24, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

Embodiment I-26. The method of any one of embodiments I-1 to I-23, wherein the metabolic disorder is Prader-Willi syndrome.

Embodiment I-27. The method of embodiment I-24, wherein the diabetes is Type I diabetes or Type II diabetes.

Embodiment I-28. The method of embodiment I-24, wherein the method treats an obesity related co-morbidity, selected from the group of: glucose intolerance, prediabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

Embodiment I-29. The method of any one of embodiments I-1 to I-28, wherein the subject has a body mass index (BMI) of 30 or greater.

Embodiment I-30. The method of any one of embodiments I-1 to I-28, wherein the subject has a BMI of 27 or greater and has one or more obesity-related co-morbidities.

Embodiment I-31. The method of any one of embodiments I-1 to I-30, wherein the subject is overweight.

Embodiment I-32. The method of any one of embodiments I-1 to I-31, wherein the subject is 18 years of age or older.

Embodiment I-33. The method of any one of embodiments I-1 to I-31, wherein the subject is 45 years of age or older.

Embodiment I-34. The method of any one of embodiments I-1 to I-31, wherein the subject is a child 0-17 years of age, inclusive.

Embodiment I-35. The method of any one of embodiments I-1 to I-34, wherein the treatment reduces body weight in the subject.

Embodiment I-36. The method of any one of embodiments I-1 to I-35, wherein the treatment reduces fat mass in the subject.

Embodiment I-37. The method of any one of embodiments I-1 to I-36, wherein the treatment increases lean mass in the subject.

Embodiment I-38. The method of any one of embodiments I-1 to I-37, wherein the treatment reduces fat mass and increases lean mass in the subject.

Embodiment I-39. The method of any one of embodiments I-1 to I-36, wherein the treatment reduces fat mass and maintains lean mass in the subject.

Embodiment I-40. The method of any one of embodiments I-1 to I-39, wherein the treatment reduces waist circumference in the subject.

Embodiment I-41. The method of any one embodiments I-1 to I-39, wherein the treatment reduces liver and/or non-liver fat mass in the subject.

Embodiment I-42. The method of any one of embodiments I-1 to I-40, wherein the treatment improves glycemic control in the subject.

Embodiment I-43. The method of any one of embodiments I-1 to I-41, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

Embodiment I-44. The method of any one of embodiments I-1 to I-41, wherein the efficacy of the treatment is assessed as about 500%, about 400%, about 300%, about 200%, about 100%, about 90%, about 80%, or about 75% of a desired “C_{trough}”.

Embodiment I-45. The method of embodiment I-44, wherein the desired “C_{trough}” is 10 μg/ml.

Embodiment I-46. The method of any one of embodiments I-1 to I-45, wherein the subject is human.

### Embodiments Set II

Embodiment II-1. A method of treating a metabolic disorder in a subject in need thereof comprising administering to the subject an ActRII antibody loading dose followed by an ActRII antibody dosage regimen.

Embodiment II-2. A method of treating a metabolic disorder in a subject in need thereof, wherein the method comprises an ActRII antibody dosage regimen, and wherein an ActRII antibody is administered to the subject about every 12 weeks.

Embodiment II-3. The method of embodiment II-2, wherein the ActRII antibody dosage regimen follows the administration of an ActRII antibody loading dose.

Embodiment II-4. The method of embodiment II-1 or II-3, wherein the ActRII antibody loading dose is about 3 mg/kg to about 50 mg/kg.

Embodiment II-5. The method of embodiment II-1 or II-3, wherein the ActRII antibody loading dose is about 10 mg/kg.

Embodiment II-6. The method of embodiment II-1 or II-3, wherein the ActRII antibody loading dose is about 30 mg/kg.

Embodiment II-7. The method of embodiment II-1 or II-3, wherein the ActRII antibody loading dose is a fixed dose of about 300 mg.

Embodiment II-8. The method of any one of embodiments II-1 to II-7, wherein the ActRII antibody dosage regimen starts about 1 day, about 2 days, about 3 days, about 4 days, about 5 days, about 6 days, about 1 week, about 2 weeks, about 3 weeks, about 4 weeks, or about 5 weeks after the loading dose of the ActRII antibody.

Embodiment II-9. The method of any one of embodiments II-1 to II-7, wherein the ActRII antibody dosage regimen comprises administration of an ActRII antibody in a dose of about 3 mg/kg to about 50 mg/kg.

Embodiment II-10. The method of any one of embodiments II-1 to II-7, wherein the ActRII antibody dosage regimen comprises administration of an ActRII antibody in a dose of about 10 mg/kg.

Embodiment II-11. The method of any one of embodiments II-1 to II-7, wherein the ActRII antibody dosage regimen comprises administration of an ActRII antibody in a dose of about 30 mg/kg.

Embodiment II-12. The method of any one of embodiments II-1 to II-11, wherein the ActRII antibody dosage regimen comprises administration of an ActRII antibody about every 4 weeks, about every 8 weeks, about every 12 weeks, or about every 16 weeks.

Embodiment II-13. The method of embodiment II-12, wherein the ActRII antibody dosage regimen comprises administration of an ActRII antibody about every 12 weeks.

Embodiment II-14. The method of any one of embodiments II-1, II-3 to II-6, and II-8 to II-13, wherein the ActRII antibody loading dose is administered intravenously.

Embodiment II-15. The method of any one of embodiments II-1, and II-3 to II-13, wherein the ActRII antibody dosage regimen is administered intravenously.

Embodiment II-16. The method of any one of embodiments II-1, II-3 to II-6, and II-8 to II-13, wherein the ActRII antibody loading dose and the ActRII antibody dosage regimen are administered intravenously.

Embodiment II-17. The method of any one of embodiments II-1 to II-7, wherein the ActRII antibody dosage regimen comprises administration of an ActRII antibody in a dose of about 200 mg to about 400 mg.

Embodiment II-18. The method of any one of embodiments II-1 to II-7, wherein the ActRII antibody dosage regimen comprises administration of an ActRII antibody in a dose of about 300 mg.

Embodiment II-19. The method of embodiment II-17 or II-18, wherein the ActRII antibody dosage regimen is administered once weekly, twice weekly, thrice weekly, or every two weeks.

Embodiment II-20. The method of embodiment II-17 or II-18, wherein the ActRII antibody dosage regimen is administered once weekly.

Embodiment II-21. The method of embodiment II-7, wherein the ActRII antibody loading dose is administered subcutaneously.

Embodiment II-22. The method of embodiment II-17 or II-18, wherein the ActRII antibody dosage regimen is administered subcutaneously.

Embodiment II-23. The method of embodiment 11-7, 11-17, or II-18 wherein the ActRII antibody loading dose and the ActRII antibody dosage regimen are both administered subcutaneously.

Embodiment II-24. The method of any one of embodiment II-1 or II-23, wherein the ActRII antibody of the loading dose comprises the amino acid sequence of SEQ ID NOS: 1-6.

Embodiment II-25. The method of any one of embodiments II-1 or II-3 to II-23, wherein the ActRII antibody of the loading dose comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto.

Embodiment II-26. The method of any one of embodiments II-1 or II-3 to II-23 wherein the loading dose of the ActRII antibody comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

Embodiment II-27. The method of anyone embodiments II-1 or II-3 to II-13, wherein the ActRII antibody of the loading dose is specific for ActRIIA and ActRIIB.

Embodiment II-28. The method of any one of embodiments II-1 to II-27, wherein the ActRII antibody dosage regimen comprises administration of an ActRII antibody comprising the amino acid sequence of SEQ ID NOS: 1-6.

Embodiment II-29. The method of any one of embodiments II-1 to II-27, wherein the ActRII antibody dosage regimen comprises administration of an ActRII antibody comprising: the amino acid sequence of SEQ ID NO: 7, or a sequence with sequence identity of at least 90% thereto, and comprising the amino acid sequence of SEQ ID NO: 8, or a sequence with sequence identity of at least 90% thereto.

Embodiment II-30. The method of any one of embodiments II-1 to II-27, wherein the ActRII antibody dosage regimen comprises administration of an ActRII antibody comprising: the amino acid sequence of SEQ ID NO: 9, or a sequence with sequence identity of at least 90% thereto, and comprising the amino acid sequence of SEQ ID NO: 10, or a sequence with sequence identity of at least 90% thereto.

Embodiment II-31. The method of any one of embodiments II-1 to II-27, wherein the ActRII antibody dosage regimen comprises administration of an ActRII antibody specific for ActRIIA and ActRIIB.

Embodiment II-32. The method of any one of embodiments II-1 to II-31, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

Embodiment II-33. The method of embodiment II-32, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

Embodiment II-34. The method of any one of embodiments II-1 to II-32, wherein the metabolic disorder is Prader-Willi syndrome.

Embodiment II-35. The method of embodiment II-32, wherein the diabetes is Type I diabetes or Type II diabetes.

Embodiment II-36. The method of embodiment II-32, wherein the method treats an obesity related co-morbidity, selected from the group of: glucose intolerance, prediabetes, insulin resistance, high triglycerides, overweight associated physical impairment, osteoporosis, renal disease, obstructive sleep apnea, sexual hormones impairment, endocrine reproductive disorders, osteoarthritis, gastrointestinal cancers, dyslipidemia, hypertension, heart failure, coronary heart disease, stroke, and/or gallstones.

Embodiment II-37. The method of any one of embodiments II-1 to II-36, wherein the subject has a body mass index (BMI) of 30 or greater.

Embodiment II-38. The method of any one of embodiments II-1 to II-36, wherein the subject has a BMI of 27 or greater and has one or more obesity-related co-morbidities.

Embodiment II-39. The method of any one of embodiments II-1 to II-38, wherein the subject is overweight.

Embodiment II-40. The method of any one of embodiments II-1 to II-39, wherein the subject is 18 years of age or older.

Embodiment II-41. The method of any one of embodiments II-1 to II-39, wherein the subject is 45 years of age or older.

Embodiment II-42. The method of any one of embodiments II-1 to II-39, wherein the subject is a child 0-17 years of age, inclusive.

Embodiment II-43. The method of any one of embodiments II-1 to II-42, wherein the treatment reduces body weight in the subject.

Embodiment II-44. The method of any one of embodiments II-1 to II-43, wherein the treatment reduces fat mass in the subject.

Embodiment II-45. The method of any one of embodiments II-1 to II-44, wherein the treatment increases lean mass in the subject.

Embodiment II-46. The method of any one of embodiments II-1 to II-45, wherein the treatment reduces fat mass and increases lean mass in the subject.

Embodiment II-47. The method of any one of embodiments II-1 to II-44, wherein the treatment reduces fat mass and maintains lean mass in the subject.

Embodiment II-48. The method of any one of embodiments II-1 to II-47, wherein the treatment reduces waist circumference in the subject.

Embodiment II-49. The method of any one embodiments II-1 to II-47, wherein the treatment reduces liver and/or non-liver fat mass in the subject.

Embodiment II-50. The method of any one of embodiments II-1 to II-48, wherein the treatment improves glycemic control in the subject.

Embodiment II-51. The method of any one of embodiments II-1 to II-49, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

Embodiment II-52. The method of any one of embodiments II-1 to II-49, wherein the efficacy of the treatment is assessed as about 500%, about 400%, about 300%, about 200%, about 100%, about 90%, about 80%, or about 75% of a desired “C_{trough}”.

Embodiment II-53. The method of embodiment II-52, wherein the desired “C_{trough}” is 10 μg/ml.

Embodiment II-54. The method of any one of embodiments II-1 to II-53, wherein the subject is human.

### Embodiments Set III

Embodiment III-1. A method of treating a metabolic disorder in a subject in need thereof, comprising administering to the subject an ActRII antibody dosage regimen, wherein the ActRII antibody dosage regimen comprises administration of an ActrII antibody in a dose of about 3 mg/kg to about 50 mg/kg, about once every 8 weeks to about once every 16 weeks, wherein the ActRII antibody dosage regimen is administered intravenously.

Embodiment III-2. The method of embodiment III-1, wherein the ActRII antibody dosage regimen follows the administration of an ActRII antibody loading dose.

Embodiment III-3. The method of any one of embodiments III-1 to III-2, wherein the ActRII antibody dosage regimen and/or the ActRII antibody of the loading dose comprises administration of an ActRII antibody comprising the amino acid sequence of SEQ ID NOS: 1-6.

Embodiment III-4. The method of any one of embodiments III-1 to III-2, wherein the ActRII antibody dosage regimen and/or the ActRII antibody of the loading dose comprises administration of an ActRII antibody comprising: the amino acid sequence of SEQ ID NO: 7, or a sequence with sequence identity of at least 90% thereto, and comprising the amino acid sequence of SEQ ID NO: 8, or a sequence with sequence identity of at least 90% thereto.

Embodiment III-5. The method of any one of embodiments III-1 to III-2, wherein the ActRII antibody dosage regimen and/or the ActRII antibody of the loading dose comprises administration of an ActRII antibody comprising: the amino acid sequence of SEQ ID NO: 9, or a sequence with sequence identity of at least 90% thereto, and comprising the amino acid sequence of SEQ ID NO: 10, or a sequence with sequence identity of at least 90% thereto.

Embodiment III-6. The method of any one of embodiments III-1 to III-2, wherein the ActRII antibody dosage regimen and/or the ActRII antibody loading dose comprises administration of an ActRII antibody specific for ActRIIA and ActRIIB.

Embodiment III-7. The method of any one of embodiments III-1 to III-6, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

Embodiment III-8. The method of embodiment III-7, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

Embodiment III-9. The method of any one of embodiments III-1 to III-6, wherein the metabolic disorder is Prader-Willi syndrome.

Embodiment III-10. The method of embodiment III-7, wherein the diabetes is Type I diabetes or Type II diabetes.

Embodiment III-11. The method of any one of embodiments III-7 to III-9, wherein the method treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

Embodiment III-12. The method of any one of embodiments III-1 to III-11, wherein the treatment reduces body weight in the subject.

Embodiment III-13. The method of any one of embodiments III-1 to III-12, wherein the treatment reduces fat mass in the subject.

Embodiment III-14. The method of any one of embodiments III-1 to III-13, wherein the treatment increases lean mass in the subject.

Embodiment III-15. The method of any one of embodiments III-1 to III-14, wherein the treatment reduces fat mass and increases lean mass in the subject.

Embodiment III-16. The method of any one of embodiments III-1 to III-14, wherein the treatment reduces fat mass and maintains lean mass in the subject.

Embodiment III-17. The method of any one of embodiments III-1 to III-16, wherein the treatment reduces waist circumference in the subject.

Embodiment III-18. The method of any one embodiments III-1 to III-17, wherein the treatment reduces liver and/or non-liver fat mass in the subject.

Embodiment III-19. The method of any one of embodiments III-1 to III-18, wherein the treatment improves glycemic control in the subject.

Embodiment III-20. The method of any one of embodiments III-1 to III-19, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

Embodiment III-21. The method of any one of embodiments III-1 to III-20, wherein the efficacy of the treatment is measured by the C_{trough} in a sample from the subject.

Embodiment III-22. The method of embodiment III-21, wherein the treatment is efficacious when the C_{trough} of the sample from the subject is at least about 500%, about 400%, about 300%, about 200%, about 100%, about 90%, about 80%, or about 75% of a desired “C_{trough}”.

Embodiment III-23. The method of embodiment III-22, wherein the desired “C_{trough}” is 10 μg/ml.

Embodiment III-24. The method of any one of embodiments III-1 to III-23, wherein the subject is human.

Embodiment III-25. A method of treating a metabolic disorder in a subject in need thereof comprising administering to the subject an ActRII antibody loading dose followed by an ActRII antibody dosage regimen.

Embodiment III-26. The method of embodiment III-25, wherein the ActRII antibody loading dose is about 3 mg/kg to about 50 mg/kg.

Embodiment III-27. The method of embodiment III-25 or III-26, wherein the ActRII antibody dosage regimen starts about 1 day, about 2 days, about 3 days, about 4 days, about 5 days, about 6 days, about 1 week, about 2 weeks, about 3 weeks, about 4 weeks, or about 5 weeks after the loading dose of the ActRII antibody.

Embodiment III-28. The method of any one of embodiments III-25 to III-27, wherein the ActRII antibody dosage regimen comprises administration of an ActRII antibody in a dose of about 3 mg/kg to about 50 mg/kg.

Embodiment III-29. The method of any one of embodiments III-25 to III-28, wherein the ActRII antibody dosage regimen comprises administration of an ActRII antibody about every 4 weeks, about every 8 weeks, about every 12 weeks, or about every 16 weeks.

Embodiment III-30. The method of any one of embodiments III-25 to III-29, wherein the ActRII antibody loading dose is administered intravenously.

Embodiment III-31. The method of any one of embodiments III-25 to III-30, wherein the ActRII antibody of the loading dose and/or the ActRII antibody of the dosage regimen comprises the amino acid sequence of SEQ ID NOS: 1-6.

Embodiment III-32. The method of any one of embodiments III-25 to III-30, wherein the ActRII antibody of the loading dose and/or the ActRII antibody of the dosage regimen comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto.

Embodiment III-33. The method of any one of embodiments III-25 to III-30, wherein the loading dose of the ActRII antibody and/or the ActRII antibody of the dosage regimen comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

Embodiment III-34. The method of any one embodiments III-25 to III-33, wherein the ActRII antibody of the loading dose and/or the ActRII antibody dosage regimen is specific for ActRIIA and ActRIIB.

Embodiment III-35. The method of any one embodiments III-25 to III-34, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

Embodiment III-36. The method of embodiment III-35, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

Embodiment III-37. The method of any one of embodiments III-25 to III-34 wherein the metabolic disorder is Prader-Willi syndrome.

Embodiment III-38. The method of embodiment III-35 wherein the diabetes is Type I diabetes or Type II diabetes.

Embodiment III-39. The method of embodiment III-35 wherein the method treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

Embodiment III-40. The method of any one of embodiments III-25 to III-39, wherein the treatment reduces body weight in the subject.

Embodiment III-41. The method of any one of embodiments III-25 to III-40, wherein the treatment reduces fat mass in the subject.

Embodiment III-42. The method of any one of embodiments III-25 to III-41, wherein the treatment increases lean mass in the subject.

Embodiment III-43. The method of any one of embodiments III-25 to III-42, wherein the treatment reduces fat mass and increases lean mass in the subject.

Embodiment III-44. The method of any one of embodiments III-25 to III-43, wherein the treatment reduces fat mass and maintains lean mass in the subject.

Embodiment III-45. The method of any one of embodiments III-25 to III-44, wherein the treatment reduces waist circumference in the subject.

Embodiment III-46. The method of any one embodiments III-25 to III-45, wherein the treatment reduces liver and/or non-liver fat mass in the subject.

Embodiment III-47. The method of any one of embodiments III-25 to III-46, wherein the treatment improves glycemic control in the subject.

Embodiment III-48. The method of any one of embodiments III-25 to III-47, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

Embodiment III-49. The method of any one of embodiments III-25 to III-48, wherein the efficacy of the treatment is measured by the C_{trough} in a sample from the subject.

Embodiment III-50. The method of embodiment III-49, wherein the treatment is efficacious when the C_{trough} of the sample from the subject is at least about 500%, about 400%, about 300%, about 200%, about 100%, about 90%, about 80%, or about 75% of a desired “C_{trough}”.

Embodiment III-51. The method of embodiment III-50, wherein the desired “C_{trough}” is 10 μg/ml.

Embodiment III-52. The method of any one of embodiments III-25 to III-51, wherein the subject is human.

The following examples are included for illustrative purposes and are not intended to limit the scope of the invention.

### EXAMPLES

### Example 1: Design of a Bimagrumab Dosage Model Based on Observed Clinical Population Pharmacokinetics (PK) and Pharmacodynamics (PD)

A pharmacokinetic and pharmacodynamic (PK/PD) model was developed to support ActRII antibody (bimagrumab) dose and frequency selection for a clinical study to lower fat body mass (FBM) and increase lean body mass (LBM) in obese subjects. Bimagrumab has been previously tested in 7 different phase I, II and III clinical studies to address different diseases using entirely different dosage regimens (see Table 1).

To perform the pharmacokinetic and pharmacodynamic (PK/PD) analysis and modeling described herein, clinical studies were selected that provided PK and PD information in the following contexts: wide dose ranges of 10-3000 mg of bimagrumab, intravenous (IV administration), varied bimagrumab dosage timing, measurements of fat body mass (FBM) and lean body mass (LBM) in response to bimagrumab, extensive study follow-up periods, and other diseases both related and unrelated to obesity.

The pharmacokinetics (PK) of bimagrumab statistically assessed and then modeled herein include the patient's exposure (i.e., volume of distribution and clearance). The pharmacodynamics (PD) of bimagrumab statistically assessed and then modeled herein include the effects on fat body mass (FBM) and lean body mass (LBM).

To determine safe and efficient dosing of bimagrumab, total fat body mass (FBM) was selected as a main pharmacodynamic (PD) indicator, while lean body mass (LBM) was selected as a secondary indicator.

### Study Objectives Included:

1. Statistically describe bimagrumab clinical study population PK characteristics
2. Statistically describe bimagrumab clinical study population PD effects on FBM and LBM
3. Statistically assess the relationship of subject-specific factors (i.e., age, body weight, dose, and disease state) on bimagrumab PK (i.e. the volume of distribution and clearance) and PD (i.e., FBM and LBM).
4. Run PK/PD simulations to predict the exposure (PK) and FBM and LBM effects (PD) of the following bimagrumab dosage regimens:
   a. 30 mg/kg bimagrumab given intravenously (IV) every 4 weeks, every 8 weeks, every 12 weeks, and every 24 weeks
   b. 50 mg/kg bimagrumab given intravenously (IV) every 12 weeks
5. Run PK/PD simulations to predict the exposure (PK) and FBM and LBM effects (PD) of two loading doses of bimagrumab
6. Run PK/PD simulations to predict to predict the exposure (PK) and FBM and LBM effects (PD) of bimagrumab dosage regimens with a loading dose at week 0 and every 12 weeks thereafter as follows:
   a. 10 mg/kg given IV at weeks 0, 4 and 16
   b. 30 mg/kg given IV at weeks 0, 4 and 16
7. Investigate the effect of body weight on bimagrumab exposure (PK) and FBM and LBM effects (PD)

TABLE 1
Clinical trials used in the PK/PD model

| | | | | | | Study | | |
|---|---|---|---|---|---|---|---|---|
| | | | | | | duration | | |
| | | | N | | | (includes | | |
| | | | patients | | | both | | |
| | | | (all | | | treatment | | |
| | | | included | | N | and follow- | DXA, | Obesity- |
| Study | Phase | Indication | in model) | Doses | Admin | up period) | days | related |
| D2201 | 2b | hip | 246 | 70, 210, 700 | 6 | 24 + 24 wk | −1, | No |
| | | fracture | | mg IV q4w | | | 83, 167 | |
| | | recovery | | | | | | |
| X2204 | 2a | chronic | 65 | 30 mg/kg | 2 | 24 wk | −1, 28, | No |
| | | obstructive | | q8w | | | 56, 84, | |
| | | pulmonary | | | | | 112, 167 | |
| | | disease | | | | | | |
| | | (COPD) | | | | | | |
| X2206 | 2a | metabolic | 16 | 30 mg/kg | 1 | 24 wk | −1, 42, | Yes |
| | | profiling | | Single | | | 70, 98, | |
| | | | | | | | 167 | |
| X2110 | 1 | intravenous/ | 91 | 210, 525, | 3 for | 12 + 8 wk | −1, 28, | Yes |
| | | subcutaneous | | 700, 1500 mg | q4wk | | 84, 140 | |
| | | bridging | | IV q4wk | 12 for | | | |
| | | | | 52., 150, 300 | q1wk | | | |
| | | | | mg SC q1wk | | | | |
| X2201 | 2a | sarcopenia | 40 | 30 mg/kg | 2 | 24 wk | −14, 14, | No |
| | | | | q8w | | | 28, 56, | |
| | | | | | | | 112, 168 | |
| E2202 | 2b | sarcopenia | 210 | 70, 210, 700 | 6 | 28 wk | −50, −14, | No |
| | | | | mg IV q4w | | | 84, 168 | |
| X2109 | 1 | cardiovascular | 68 | 10 mg/kg | 6 | 48 wk | −14, 84, | Yes |
| | | safety study | | q4w | | | 168, 322 | |

### Example 2: Modeling Methods

### Software

For PK parameter estimation and diagnostic plots, a validated version Monolix Suite 2019R2 was used (Monolix version 2019R2. Antony, France: Lixoft SAS, 2019.). For the human PK/PD simulations, Simulx (Monolix Suite 2019R2), a validated version of the lixoftConnectors 2019.2, mlxR 4.2.0 (Lavielle M. mlxR: Simulation of longitudinal data. Version 4.0.0. 2019. www.simulx.webpopix.org.) and a validated version of R 4. 1.1 (R Development Core Team. R: A language and environment for statistical computing. R Foundation for Statistical Computing, Vienna, Austria. ISBN 3-900051-07-0, 2008, www.Rproject.org.) were used.

### Guidelines

The data analysis was in accordance with the FDA Guidance for Industry on Population Pharmacokinetics (FDA 1999), the EMA guideline on PopPK analyses (EMA 2007]), and the LYO-X standard operating procedures (SOP) from the QMS Version 3.74.

### Data Programming and QC

Individual PK, dual-energy x-ray absorptiometry (DXA), demographics data and dosage information were used. Data from all studies were merged into a single PK/PD analysis data set. An automated quality control (QC) check of the analysis data sets was performed using a script to ensure formal correctness of the data set for population PK/PD modeling with Monolix. The QC output indicated no errors in the data set programming. The analysis data set included 743 subjects. The total number of observations was 10,006, out of these 688 (6.9%) were below the limit of quantification.

### Handling of Missing Data and Outliers

No outliers were identified via visual inspection of the data and no further steps were taken to identify and handle outliers. The analysis data set had no missing values. Data below the limit of quantification (BLQ) were included in the population PK analysis using the censored methodology available with Monolix.

### Parameter Estimation

Population parameters were estimated using the SAEM algorithm implemented in Monolix. The number of iteration steps, k1 and k2, were set to 700 and 200. It was confirmed with the SAEM convergence plots that the number of iteration steps was sufficiently large to have convergence to stable parameter estimates in all cases. The initial estimates for the fixed effects parameters were determined by fitting the model manually to the data using the ‘Check initial fixed effects’ functionality in Monolix. The initial standard deviations of the random effects were set to 1. In the consecutive modeling steps, the parameter estimates from the previous model were used as initial estimates for the next modeling step. The standard errors of the parameter estimates were derived from the Fisher information matrix using stochastic approximation. For the individual parameters, the conditional mean and standard deviation were computed. For the stopping rule of the individual parameter estimation, the MCMC evaluation length (L_mcmc) was set to 50. The −2 log-likelihood (−2LL) was computed using importance sampling with a Monte Carlo chain length of 15′000. It was confirmed via the standard error of the −2LL estimates that this Monte Carlo chain was sufficiently long.

### Model Development Approach

For the development of the base model diagnostic plots, the standard error of the parameter estimates and the −2LL were used as selection criteria.

For covariate model building, the correlation plots of individual parameter estimates versus covariates were used to select covariates for testing. Covariates with a p-value>0.01 were retained in the model.

### Diagnostic Plots

All diagnostic plots were constructed using Monolix. The visual predictive checks (VPCs) were based on 500 simulations with the model and the design structure of the observed data (i.e., dosing, timing, covariates and number of samples). The VPCs compared the median and 10th and 90th percentiles of the simulated data to the corresponding percentiles of the observed data. For some VPC plots the 90 percent (%) prediction interval of the simulated percentiles was shown in addition. Binning was adjusted manually to get reasonable spaced bins with sufficient number of observations within each bin. Details on the construction of the VPCs are given in chapter 9.7 of Lavielle 2014 (M. Lavielle. Mixed Effects Models for the Population Approach: Models, Tasks, Methods and Tools. Chapman and Hall/CRC, 2014).

The observed versus predicted plots, the boxplots of the standardized random effects, the histograms of the individual parameters, and the covariate versus parameter plots and the residual plots were constructed using random sampling from the conditional distributions of the individual parameters (Lavielle and Ribba 2016). BLQ data was included in the diagnostic plots by sampling the BLQ data from the conditional distribution (A. Samson, M. Lavielle, F. Mentre. Extension of the SAEM algorithm to left-censored data in nonlinear mixed-effects model: Application to HIV dynamics model. Computational Statistics & Data Analysis. 2006, 51(3):1562-74).

For the residual plots the individual weighted residuals (IWRES), the population weighted residuals (PWRES) and the normalized prediction distribution errors (NPDE) were used. The IWRES are estimates of the standardized residual based on individual predictions, the PWRES are the normalized difference between the observations and their mean and the NPDE are a nonparametric version of PWRES based on a rank statistic (E. Comets, K. Brendel, and F. Mentré. Computing normalized prediction distribution errors to evaluate nonlinear mixed-effect models: the npde add-on package for R. Computer Methods and Programs in Biomedicine. 2008, 90(2):154-166). The residual plots show in addition the 10%, 50% and 90% quantiles. Details on the definition and computation of the residuals are given in (Lavielle 2014).

### Simulation Methodology

PK/PD simulations were done in R using the Simulx mlxR library. With mlxR the simulation model file was automatically generated using the Monolix results as an input to the function monolix2simulx( ). Simulations were done with and without between-patient variability. The observational was set to zero. Simulations included the covariate effects. For each simulation scenario (dose and patient group) 2500 individuals were simulated, and the summary statistics was calculated. For the calculation of the area under the curve (AUC), the trapezoidal rule was used on the simulated PK parameters.

### Structural Model

The model to describe bimagrumab PK in humans was two-compartmental with first-order clearance and a Michaelis-Menten clearance from the central compartment. This was in accordance with the known PK properties of monoclonal antibodies binding to a cell surface target undergoing target mediated drug disposition (TMDD). The equations were:

$$\frac{d C c}{d t} = - Cl / {V}_{c} C c - \frac{V max C c}{K m + C c} - \frac{Q}{{V}_{c}} ( C c - Cp )$$

$$\frac{d C p}{d t} = \frac{Q}{{V}_{p}} ( C c - C p )$$

where Cc was the bimagrumab concentration in the central compartment and Cp the concentration in the peripheral compartment.

FBM was described with a turnover model with a production rate k_{FBM-in} and a first order elimination rate k_{FBM-out}. LBM was described with a turnover model with a production rate k_{LBM-out} and a first order elimination rate k_{LBM-out}:

$$\frac{d F B M}{d t} = {E}_{FBM - in} {k}_{FBM - i n} - {E}_{FBM - out} {k}_{FBM - out} FBM$$

$$\frac{d L B M}{d t} = {E}_{LBM - i n} {k}_{FLM - i n} - {k}_{FLM - out} F L M$$

The effect of bimagrumab on FBM was modeled by linking serum bimagrumab concentration with either an effect on the production rate (E_{FBM-in}) or the elimination rate (E_{FBM-out}). The effect on LBM was modelled by linking the serum bimagrumab concentration with an effect on the production rate (E_{LBM-in}). Three different effect models were tested. The linear model for the effect on the out rates was written as:

$${E}_{out} = 1 + slope Cc .$$

The exponential model for the effect on the out rates was written as:

$${E}_{o u t} = 1 - {I}_{max} ( {e}^{- k C c} - 1 ) ,$$

and for the effect on the in rates as:

$${E}_{i n} = 1 + {I}_{max} ( 1 - {e}^{- k C c} ) ,$$

The Emax model for the effect on the out rates was written as:

$${E}_{o u t} = 1 + \frac{E max C {c}^{γ}}{E C 5 {0}^{γ} + C {c}^{γ}}$$

and for the effect on the in rates as:

$${E}_{i n} = 1 - \frac{E max C {c}^{γ}}{E C 5 {0}^{γ} + C {c}^{γ}} .$$

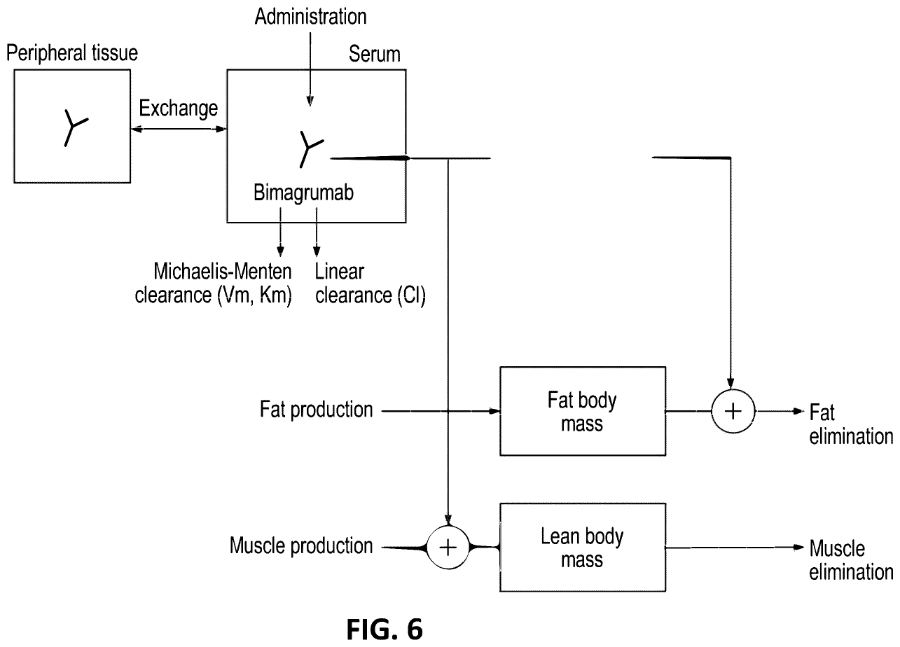

The PK/PD model schematic is shown in FIG. 6.

### Statistical Model

Individual parameters were modeled as random variables, with log-normal distributions. The equation for an individual parameter was:

$${φ}_{i} = {φ}_{p o p} \cdot {e}^{η}$$

where φₚₒₚ was the population typical parameter, 77 was a random variable with mean 0 and standard deviation ω. A combined error model was used for modeling the bimagrumab, FBM and LBM observations:

$${y}_{o b s} = {y}_{p r e d} + ( a + b \cdot {y}_{p r e d} ) \cdot ε$$

where y_{obs} was the observation, y_{pred} the model prediction, E was an independent random variable, normally distributed with mean 0 and variance 1. The parameter a described the standard deviation of the constant error and the parameter b the proportional coefficient. Continuous covariates were modelled with the equation:

$${φ}_{i} = {φ}_{p o p} \cdot {( \frac{{Ω}_{i , t}}{{Ω}_{p o p}} )}^{β} \cdot {e}^{η} ,$$

where Wᵢ was the covariate of the subject i and Wₚₒₚ was the typical population covariate. Categorical covariates were modelled with the equation:

$${φ}_{i} = {φ}_{p o p} \cdot {e}^{β} \cdot {e}^{η} ,$$

where 1_{Wi=W1}=1 if the individual covariate was in the category and 1_{Wi=W1}=0 otherwise. The typical population value for body weight was set to 70 kg.

### PK/PD Model Development

Model development was carried out in three steps. In the first step, a base PK model was developed followed by the PK covariate model development. In the second step, the FBM PD model was developed, and in the third step the LBM PD model was added. Thus, the final model linked dose with PK, and the effects of bimagrumab on FBM and LBM.

### A. PK Model Development

Based on the known PK properties of an immunoglobulin IgG1 molecule and that the binding to a cell surface receptor can result in target-mediated drug disposition (TMDD), as was evident from the PK clinical study profiles, a two-compartmental PK model with linear clearance and with Michaelis-Menten clearance was tested. This model described the data as judged by the diagnostic plots and no further refinements of the base model was needed. The correlation plots between body weight, clearance and central volume of distribution showed a correlation value of >0.3. These covariate relationships were tested and were found to be statistically significant (p<10⁻¹⁵). Even though the −2 log-likelihood (−2LL) was larger for the model with covariates, it was selected for the highly significant covariate coefficients. The correlation plots showed no effects between study, age and gender with any of the PK parameters. Thus, the final covariate model included the effect

### B. FBM Model Development

A turnover model for FBM was used with the FBM at baseline as a regressor. The use of FBM at baseline as a regressor was done because of the large between-patient variability in the baseline values that would result in population predictions far from the individual predictions. The effect of bimagrumab was either put on the FBM input rate or the elimination rate. A sigmoid effect model and an exponential effect model were tested. The model with an effect on the output rate had a better −2LL. Closer inspection of the data showed that in some patients the FBM increased over the course of the treatment. For this reason, a normally distributed parameter describing the maximal effect (max_effect) was tested that would allow negative values to describe increasing FBM. The model with an exponential effect on the output FBM rate was selected based on the lowest −2LL and the ability to describe increasing FBM.

### C. LBM Model Development

A turnover model for the FBM was used and the bimagrumab effect was put on the LBM input rate. The effect on the LBM elimination rate was not tested because the mechanism of action of bimagrumab suggests modulation of muscle growth rather than muscle elimination. For the same reason as for the FBM model, LBM at baseline was used as a regressor variable. The effect model tested was an exponential model. This model provided a good model fit as judged by the diagnostic plots. In order to capture possible relationships between FBM and LBM the correlation between the maximal effects on FBM and on LBM, the correlation between the potency on FBM and LBM and the correlation between the elimination rates of FBM and LBM were included into the model. While this model had a worse −2LL it was decided to keep these correlations to ensure that for simulations only realistic combinations of maximal effect, turnover rates and potencies for FBM and LBM were used.

### Example 3: Observed Patient Statistics from 7 Clinical Studies of Bimagrumab

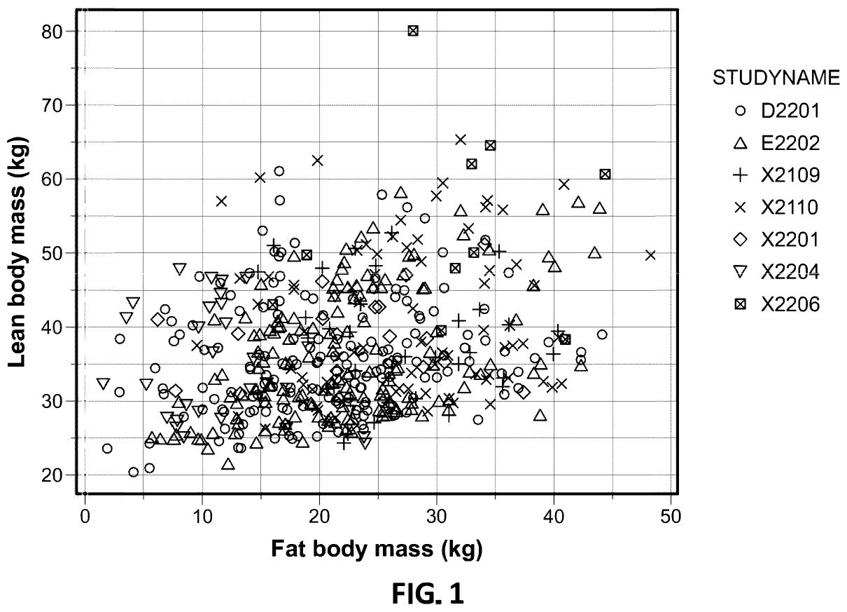

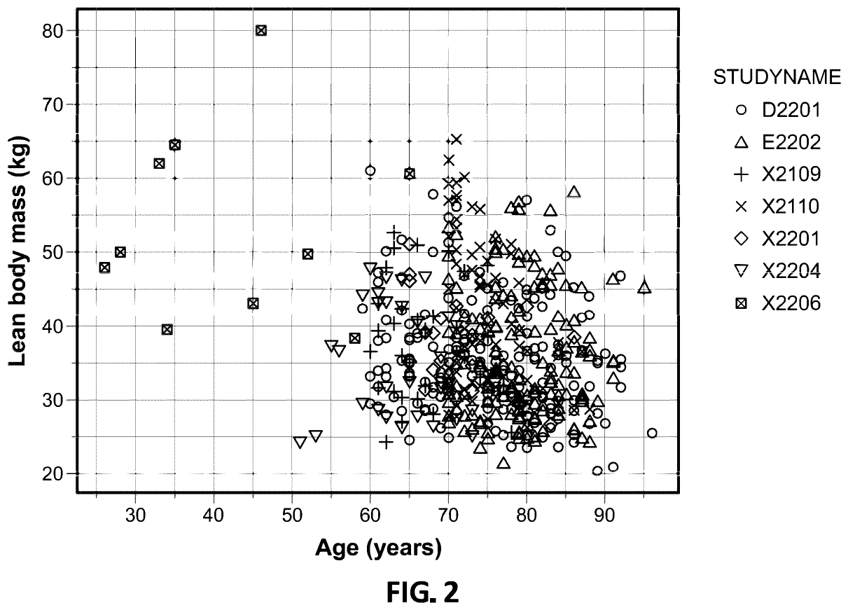

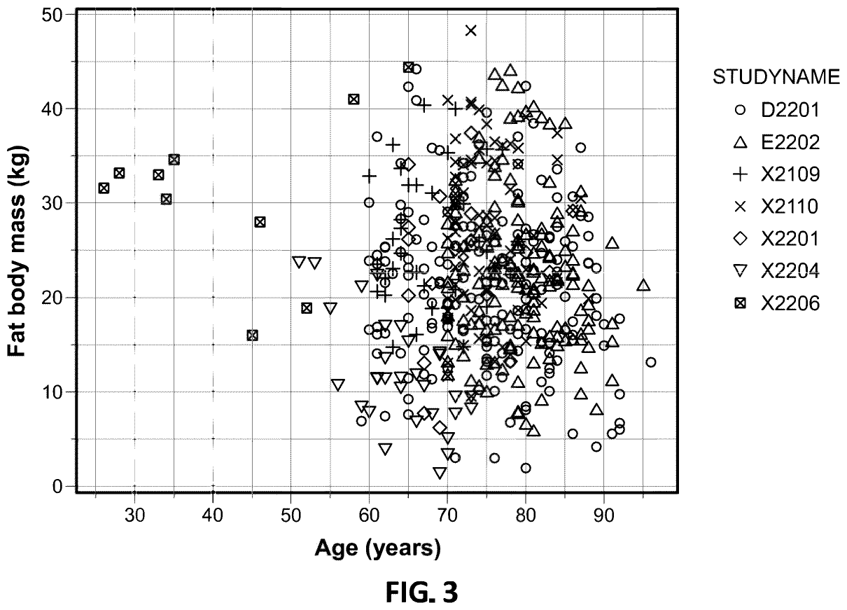

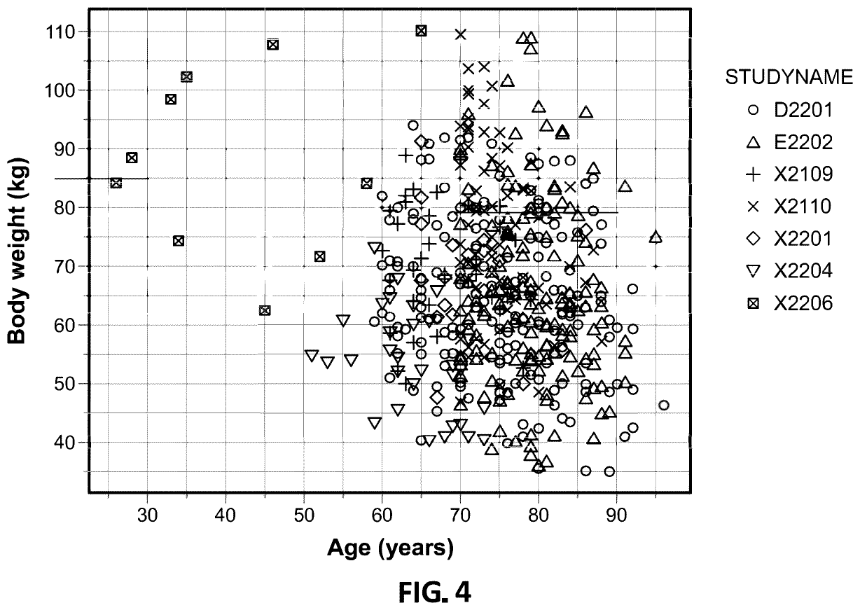

7 clinical studies of bimagrumab with entirely different dosage regimens, as described in Example 1, were statistically analyzed for PK and PD characteristics. It was observed that individual lean body mass (LBM) increased with fat body mass (FBM) at baseline (FIG. 1). Lean body mass, and possibly fat body mass, at baseline also decreased with age (FIG. 2 and FIG. 3). Accordingly, body weight appeared to decrease with age (FIG. 4). The age range across all included studies was 26-96 years, the body weight range was 35-110 kg, the lean body mass range was 20.3-80 kg, and the fat body mass range 1.5-48.3 kg. 42% of all subjects were male. Tables 2 and 3 below show the summary statistics for the age, body weight, lean body mass, fat body mass, BMI, and sex by study and for the total PK/PD analysis data set.

TABLE 2
Age versus body weight at baseline

| | | | | Fat body | Lean body | |
|---|---|---|---|---|---|---|
| Study | N | Age | Body weight | weight | weight | BMI |
| D2201 | 173 | 75.4, 8.73, | 64.1, 13.38, | 20.9, 8.60, | 35.5, 7.76, | 24.5, 3.91, |
| | | (59.0-96.0) | (35.0-94.1) | (1.9-44.2) | (20.3-61.0) | (15.4-34.7) |
| E2202 | 150 | 79.3, 5.61, | 65.1, 15.53, | 22.2, 8.18, | 35.5, 8.62, | 24.1, 3.30, |
| | | (70.0-95.0) | (35.8-108.9) | (5.7-43.9) | (21.2-57.9) | (15.2-32.4) |
| X2109 | 35 | 67.8, 5.61, | 70.5, 10.18, | 26.4, 6.94, | 37.5, 7.77, | 27.4, 3.40, |
| | | (60.0-79.0) | (50.0-89.0) | (14.8-40.4) | (24.3-52.7) | (19.8-34.0) |
| X2110 | 77 | 74.8, 4.43, | 75.8, 14.66, | 27.3, 8.29, | 41.0, 10.22, | 28.0, 3.76, |
| | | (70.0-88.0) | (46.9-109.7) | (9.5-48.3) | (26.2-65.3) | (20.0-34.2) |
| X2201 | 19 | 71.6, 6.34, | 69.0, 10.82, | 22.9, 8.29, | 38.2, 6.21, | 24.9, 3.70, |
| | | (65.0-86.0) | (47.7-91.4) | (6.2-37.4) | (26.7-51.1) | (18.0-30.9) |
| X2204 | 31 | 64.0, 5.73, | 53.9, 8.95, | 12.1, 5.81, | 35.4, 7.78, | 19.4, 2.09, |
| | | (51.0-73.0) | (40.5-73.4) | (1.5-23.9) | (24.4-48.0) | (15.2-23.4) |
| X2206 | 10 | 42.2, 13.15, | 88.5, 16.14, | 31.1, 8.71, | 53.5, 13.09, | 29.6, 4.23, |
| | | (26.0-65.0) | (62.5-110.3) | (16.0-44.4) | (38.3-80.0) | (24.2-37.7) |
| All | 495 | 74.4, 9.32, | 66.7, 15.04, | 22.4, 8.92, | 37.0, 9.03, | 24.9, 4.15, |
| studies | | (26.0-96.0) | (35.0-110.3) | (1.5-48.3) | (20.3-80.0) | (15.2-37.7) |

TABLE 3
Age versus body weight at baseline by sex

| Study | N male/female | Percent male/female |
|---|---|---|
| D2201 | 55/118 | 32/68 |
| E2202 | 67/83 | 45/55 |
| X2109 | 13/22 | 37/63 |
| X2110 | 36/41 | 47/53 |
| X2201 | 13/6 | 68/32 |
| X2204 | 15/16 | 48/52 |
| X2206 | 8/2 | 80/20 |
| All studies | 207/288 | 42/58 |

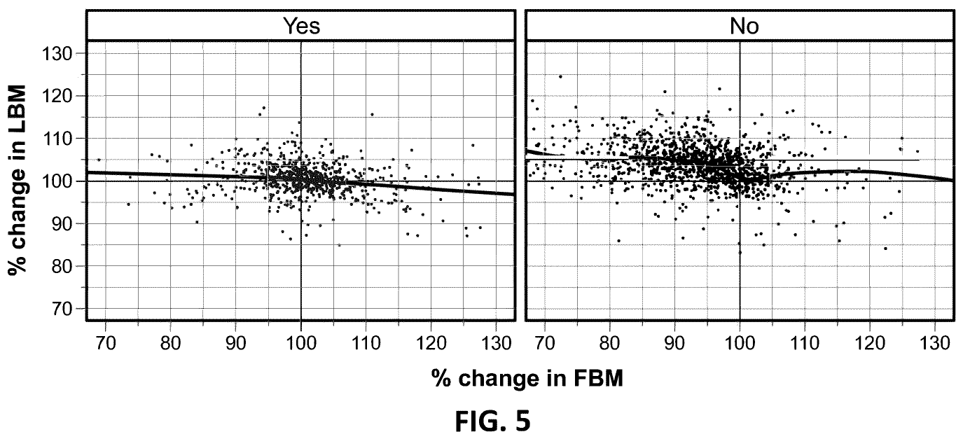

FIG. 5 shows the relationship between the percent change in LBM and FBM relative to baseline. The change in LBM was in a range of approximately 90-120% and the change in FBM in a range of 70-120%. For treated patients a reduction in FBM from 100 to 95% was linked with an increase in LBM from 100 to 105% on average, as seen from a spline fit. A further reduction of FBM from 95% to 70% was not associated with any further change in LBM, which remained at 105% on average. There was a placebo response which was on average much stronger in the relative change in FBM and almost absent in LBM, as seen from the spline fit.

Bimagrumab had typical IgG1 properties with a central volume of distribution of 3.16 L, a peripheral volume of distribution of 5.33 L and a terminal half-life of 22.46 days. Binding to ActRII resulted in target mediated drug disposition (TMDD) with a Km of 0.0414 mg/mL and a Vₘₐₓ of 0.607 mg/L/day.

Fat body mass (FBM) had a natural elimination half-life of 322 days compared to 25 days for lean body mass (LBM). Thus, the onset of effect as well as the return to baseline after treatment was expected to be much faster for LBM than for FBM. The EC₅₀ for the effect of bimagrumab on FBM was 0.76 mg/mL and on LBM it was 0.38 mg/mL. Thus, bimagrumab acted on FBM and LBM in a similar concentration range. The correlation between the individual EC₅₀ on LBM and FBM was strong (R=0.919), while the correlation between the individual maximal effects on LBM and FBM was weak (R=0.239). The correlation between the LBM and FBM turnover rates was absent. Thus, for an individual subject, the LBM and FBM turnover can be different, but the effect of bimagrumab is expected to be strongly interdependent for fat and muscle mass. There was no effect of age, sex, body weight on any of the FBM and LBM PD parameters. Further, the response was the same for subjects with hip fracture but who were otherwise healthy, patients with COPD, subjects with sarcopenia or subjects with metabolic disorders.

Body weight was found to be a statistically significant covariate on clearance (b=0.619) and the central volume of distribution (b=0.661). This body weight effect largely agrees with the one expected for an IgG1 molecule. Sex and age did not show any effect on exposure. Between-patient variability was as expected and low for the parameters related to PK after IV administration and non-specific clearance.

### Example 4: Observed Clinical Study PK and PD Statistics Versus Dosage Regimen Model Predictions

The dosage regimen model was assessed and determined to accurately describe the observed clinical data without any bias or other deviations.

### PK Model Assessment

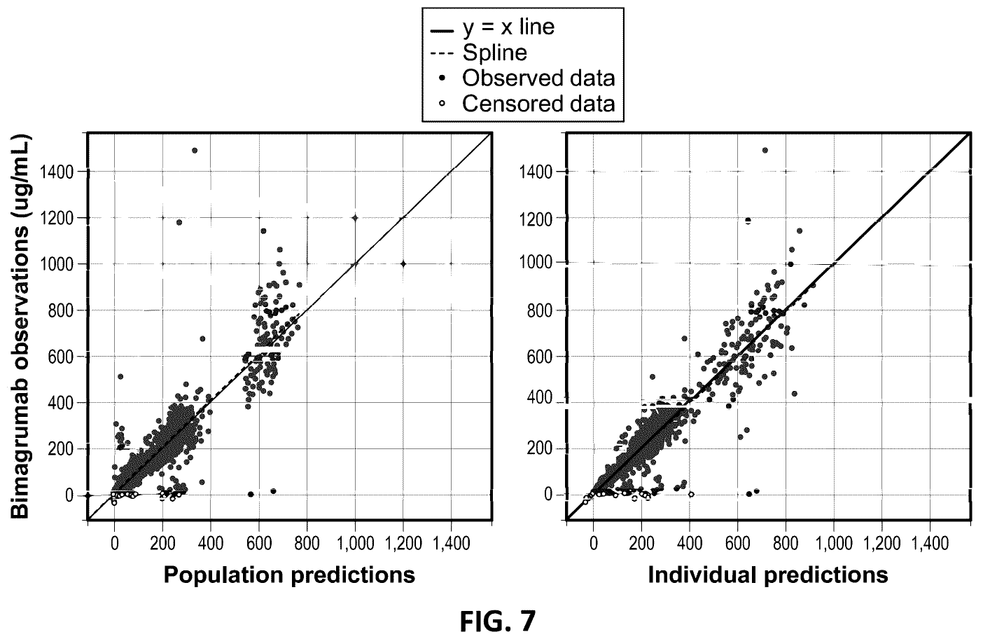

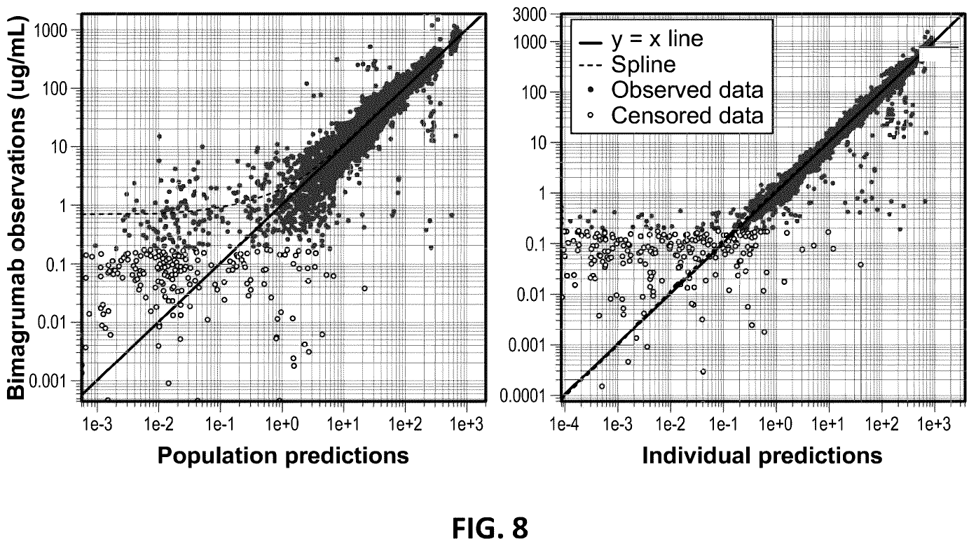

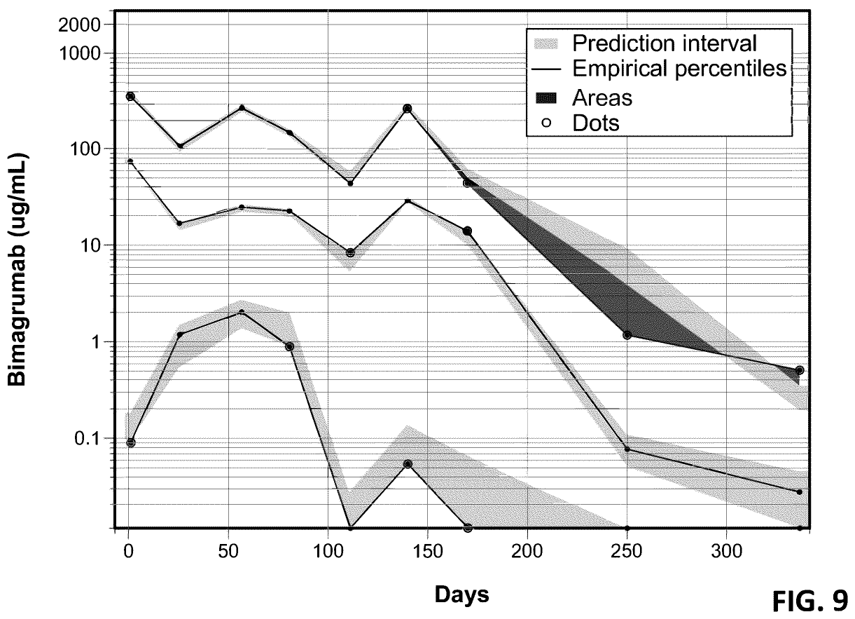

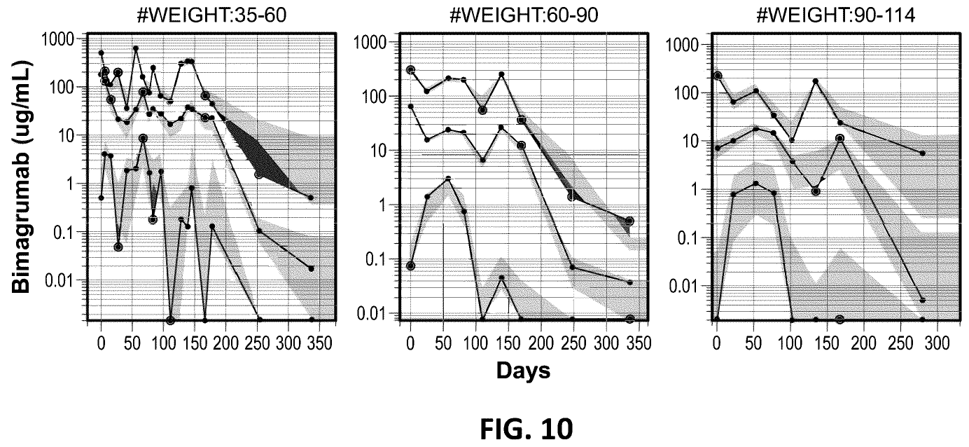

The Goodness-of-Fit (GoF) plot was overall good without bias except for clinical concentrations of bimagrumab below 1 ug/mL (FIGS. 7 and 8). The visual predictive checks (VPC) across all clinical trial data (FIG. 9) as well as the VPC by body weight groups of the clinical populations showed that the model accurately described the median and the 10^{th} and 90^{th} percentiles (FIG. 10). The histogram of the simulated individual clinical study patient parameters showed that the estimated parameters agreed with the statistical model except for Km, which appeared to have a few values much larger than the typical population value. There was no trend between the individual random effects and sex, body weight or age of the clinical study patients demonstrating that the appropriate covariate model was used.

### FBM and LBM Model Assessment

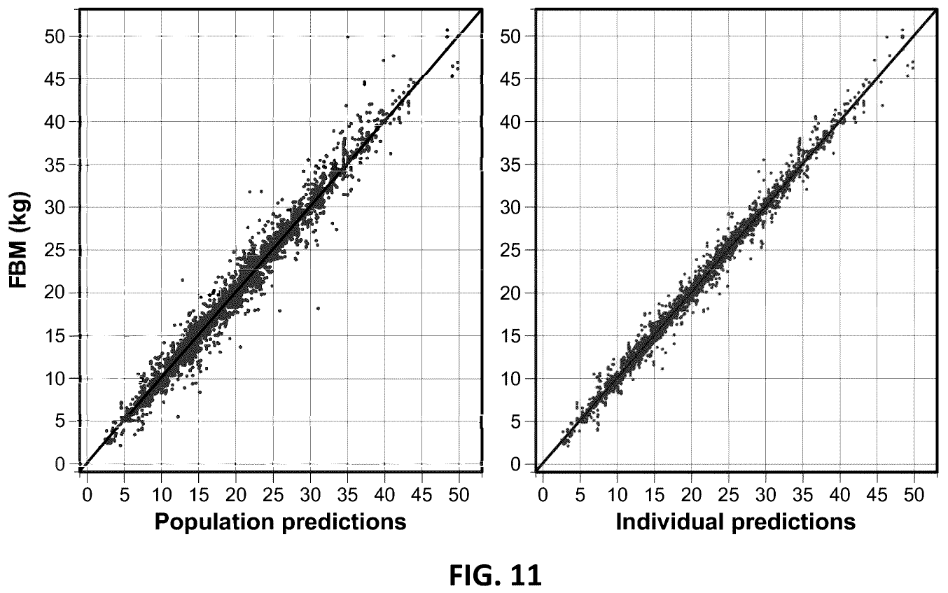

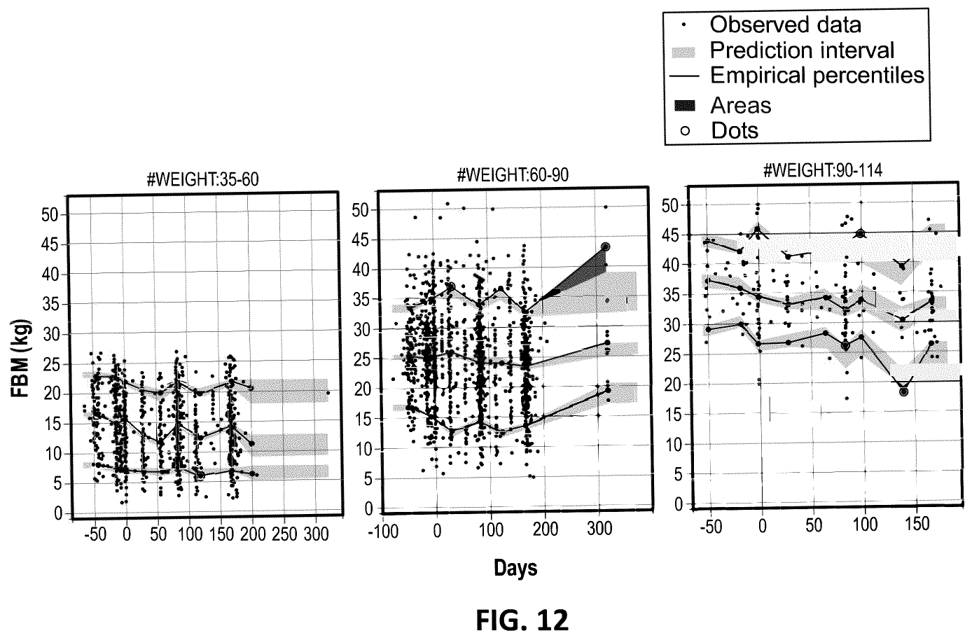

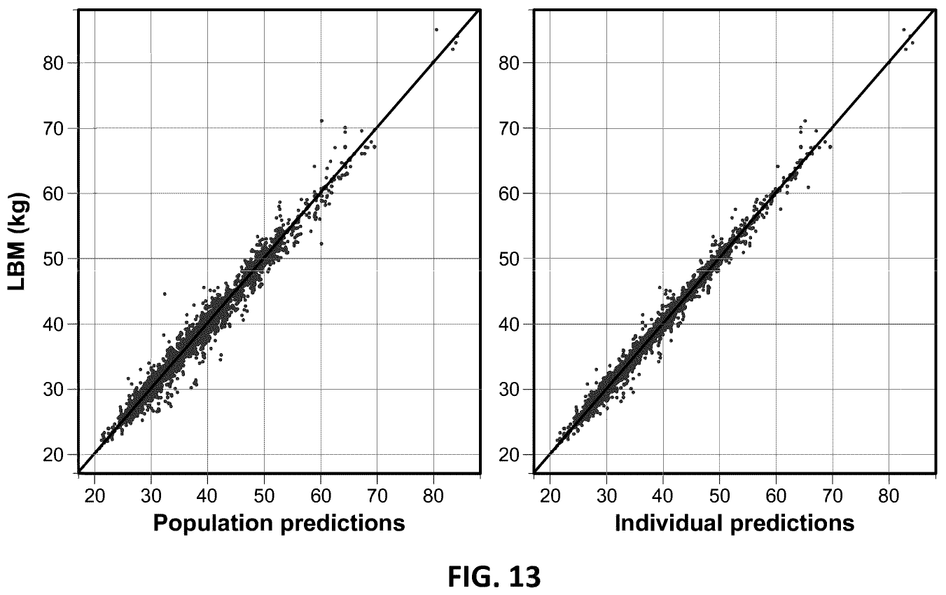

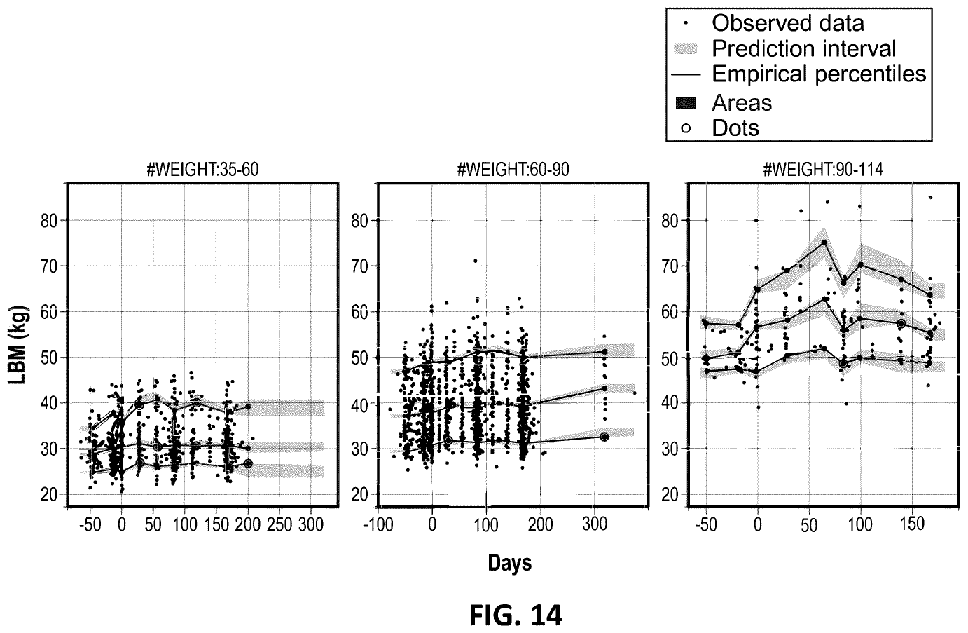

The FBM Goodness-of-Fit (GoF) plot and the VPCs plots (FIGS. 11 and 12) had no bias between the observed clinical patient population and individual FBM values and the model predicted FBM values. The LBM Goodness-of-Fit (GoF) plot and the VPCs plots (FIGS. 13 and 14) also had no bias and indicated that the model described LBM without any bias.

Simulated individual parameters also showed that the estimated parameters of the predictive PK/PD model agreed with statistically derived parameters.

In summary, the model assessment indicated that the final model described well the observed data without any bias or other deviations. The population PK/PD model was therefore considered to be appropriate to predict the exposure and effect on FBM and LBM of bimagrumab for different dosage regimen scenarios.

### Example 5: Model Predictions of Bimagrumab Dosage Regimen on Exposure (PK) and FBM and LBM (PD)

The predicted effective fat body mass half-life was simulated relative to bimagrumab concentration. The apparent saturation concentration of the ActRII receptor based on this curve is about 10 μg/ml. Thus, it is advantageous to maintain the serum concentration of bimagrumab at or above 10 μg/ml.

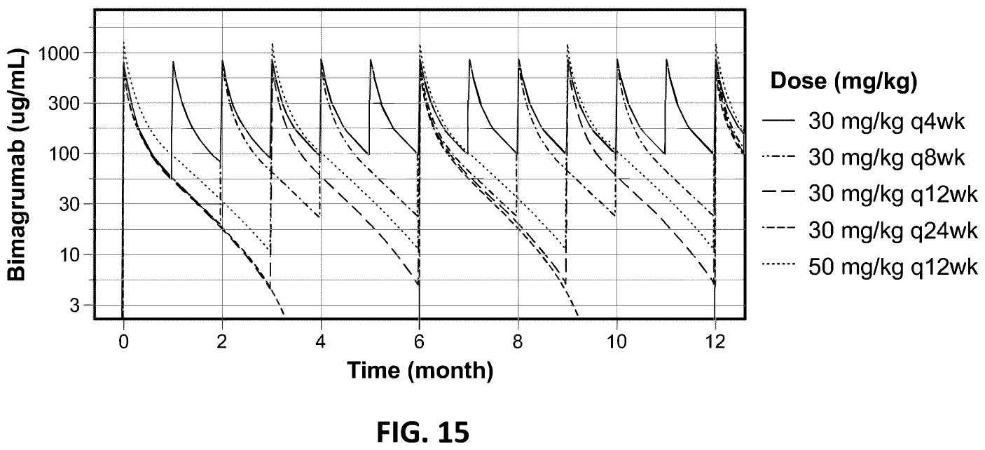

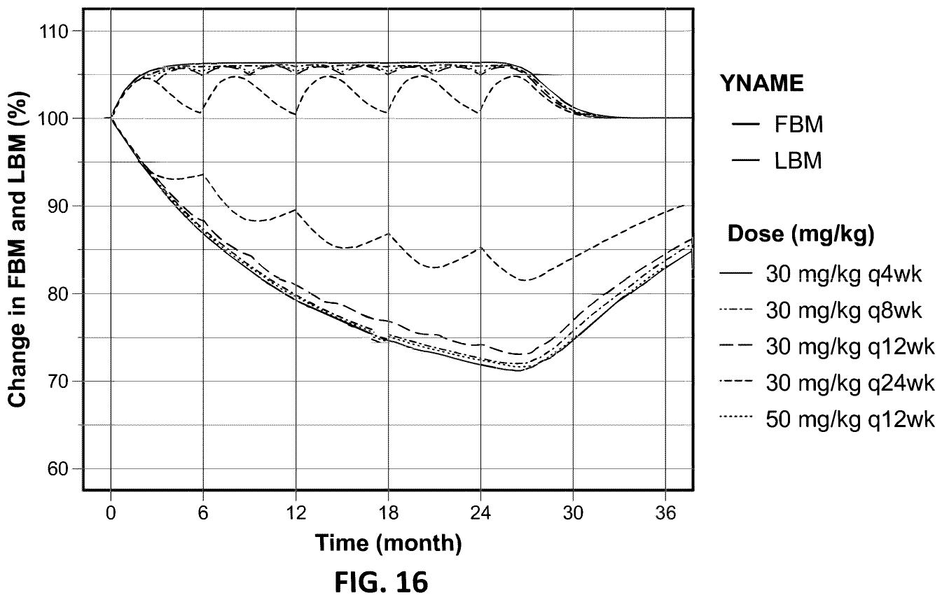

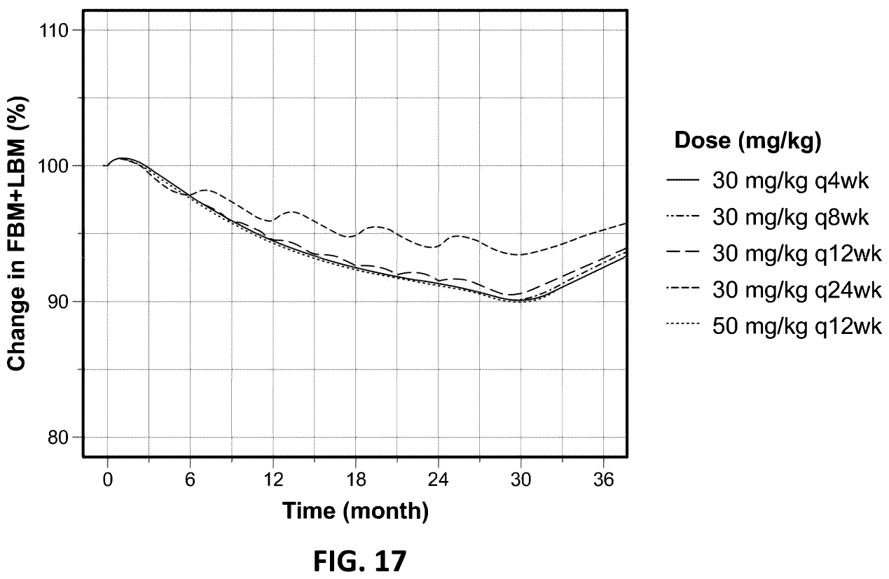

PK/PD simulations were run to predict the exposure and effect of 30 mg/kg bimagrumab given by IV every 4, 8, 12 or 24 weeks. 50 mg/kg given every 12 weeks was also tested. The treatment was modeled for 24 months. FIG. 15 shows the median exposure for the IV dosage regimen and FIG. 16 shows the effect on LBM and FBM as percent (%) change from baseline. FIG. 17 shows the effect of bimagrumab dosage on the FBM+LBM. Table 4 shows the summary statistics for the predicted effect FBM and LBM effects of bimagrumab dosage at 6, 12, 18 and 24 months.

TABLE 4
Predicted FBM and LBM response to bimagrumab
dosage (% relative to baseline)

| | | FBM Median | LBM Median |
|---|---|---|---|
| | | (10th, 90th | (10th, 90th |
| Week | Dosing | percentiles) | percentiles) |
| 24 | 30 mg/kg q4wk | 86.86 (72.02, 86.86) | 106.2 (99.38, 106.2) |
| | 30 mg/kg q8wk | 87.54 (71.96, 87.54) | 105.8 (99.78, 105.8) |
| | 30 mg/kg q12wk | 88.24 (73.48, 88.24) | 104.8 (99.91, 104.8) |
| | 30 mg/kg q24wk | 93.58 (83.74, 93.58) | 100.6 (99.98, 100.6) |
| | 50 mg/kg q12wk | 87.35 (72.25, 87.35) | 105.3 (99.61, 105.3) |
| 48 | 30 mg/kg q4wk | 79.22 (60.41, 79.22) | 106.3 (99.37, 106.3) |
| | 30 mg/kg q8wk | 79.93 (60.33, 79.93) | 105.9 (99.78, 105.9) |
| | 30 mg/kg q12wk | 81.05 (62.22, 81.05) | 104.9 (99.91, 104.9) |
| | 30 mg/kg q24wk | 89.53 (75.62, 89.53) | 100.6 (99.98, 100.6) |
| | 50 mg/kg q12wk | 79.7 (61.16, 79.7) | 105.3 (99.6, 105.3) |
| 72 | 30 mg/kg q4wk | 74.63 (55.33, 74.63) | 106.3 (99.37, 106.3) |
| | 30 mg/kg q8wk | 75.31 (55.82, 75.31) | 105.9 (99.78, 105.9) |
| | 30 mg/kg q12wk | 76.91 (57.04, 76.91) | 104.9 (99.91, 104.9) |
| | 30 mg/kg q24wk | 86.87 (71.33, 86.87) | 100.6 (99.98, 100.6) |
| | 50 mg/kg q12wk | 75.21 (56.17, 75.21) | 105.3 (99.6, 105.3) |
| 96 | 30 mg/kg q4wk | 71.87 (52.92, 71.87) | 106.3 (99.37, 106.3) |
| | 30 mg/kg q8wk | 72.6 (53.32, 72.6) | 105.9 (99.78, 105.9) |
| | 30 mg/kg q12wk | 74.25 (54.71, 74.25) | 104.9 (99.91, 104.9) |
| | 30 mg/kg q24wk | 85.21 (69.05, 85.21) | 100.6 (99.98, 100.6) |
| | 50 mg/kg q12wk | 72.58 (53.84, 72.58) | 105.3 (99.6, 105.3) |

### Effects of Loading Dose

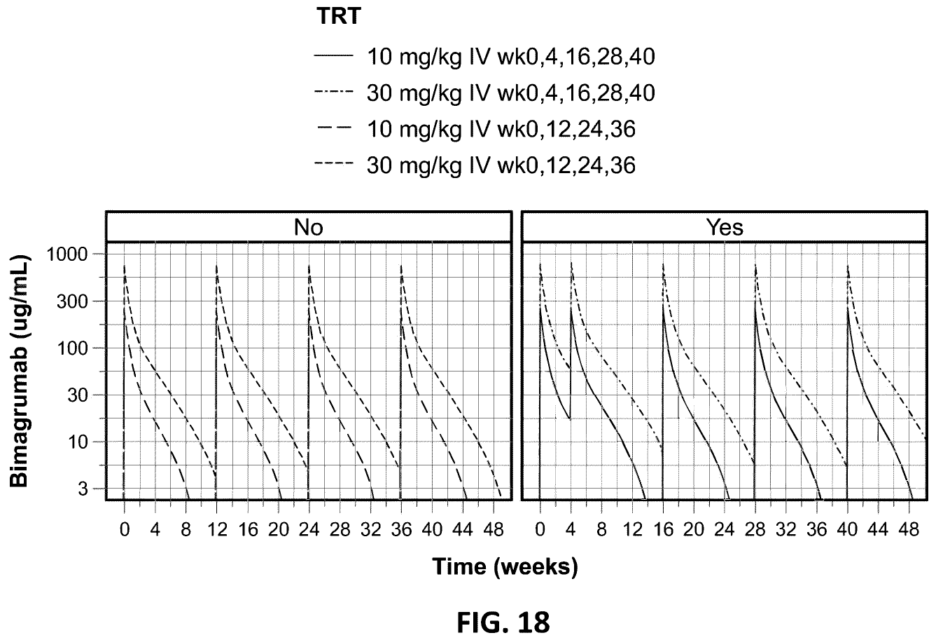

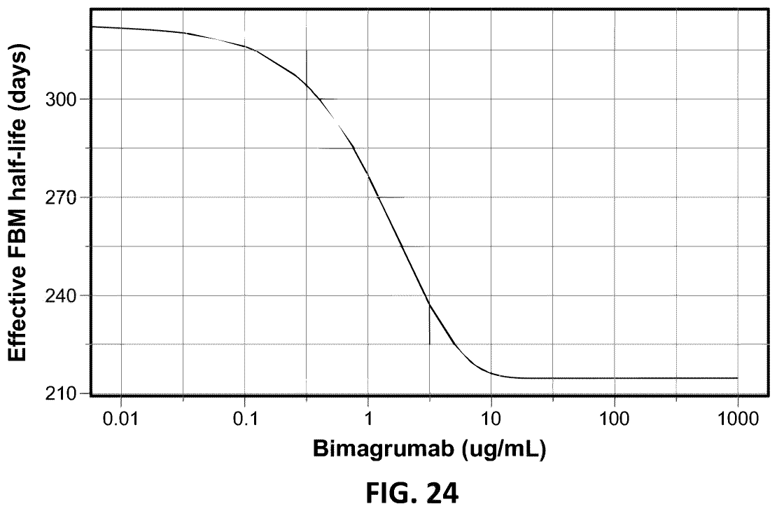

PK/PD simulations were then run to predict the exposure and effect of 10 mg/kg or 30 mg/kg bimagrumab given by IV every 12 weeks, each with and without a loading dose of the same concentration. FIG. 18 shows the median exposure for the dosage regimens with and without a loading dose. Notably, the addition of the loading dose to the dosage regimen of 30 mg/kg maintains the C_{trough} above 10 μg/ml (as shown in FIG. 18), which is predicted to the saturation concentration of the ActRII receptor (FIG. 24). Thus, the dosage regimen of 30 mg/kg every 12 weeks with at least one loading dose is predicted to enable continuous fat loss.

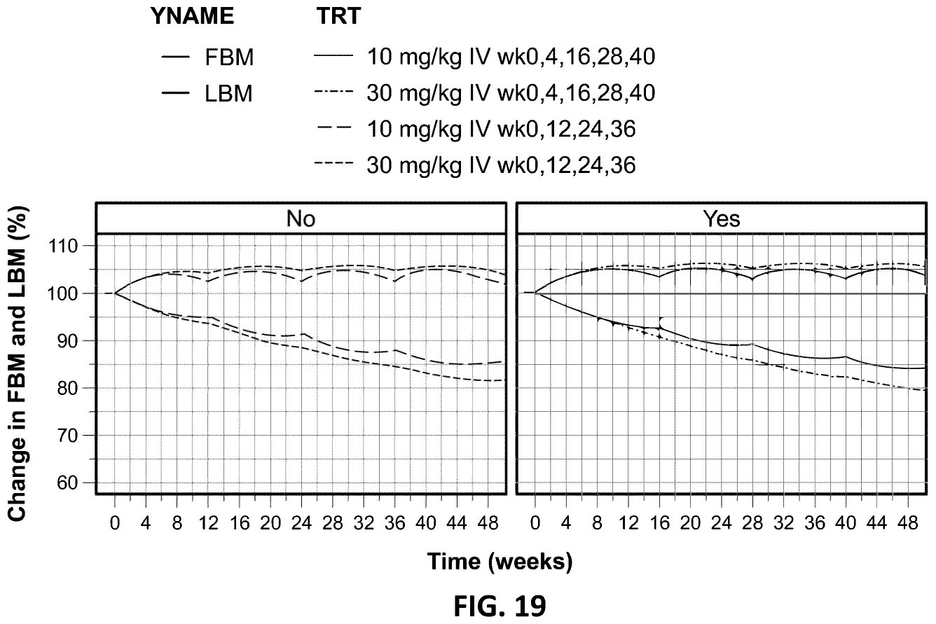

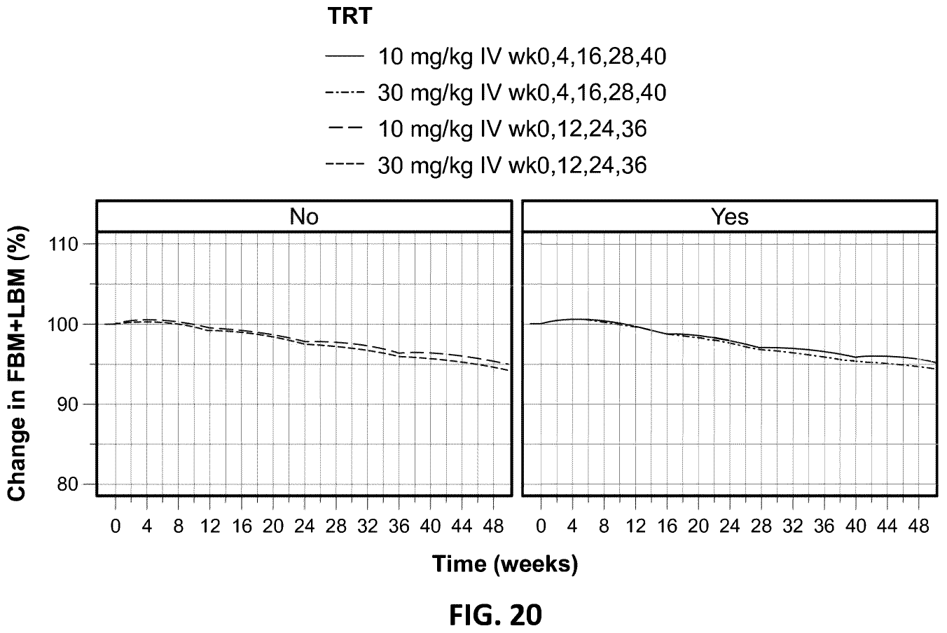

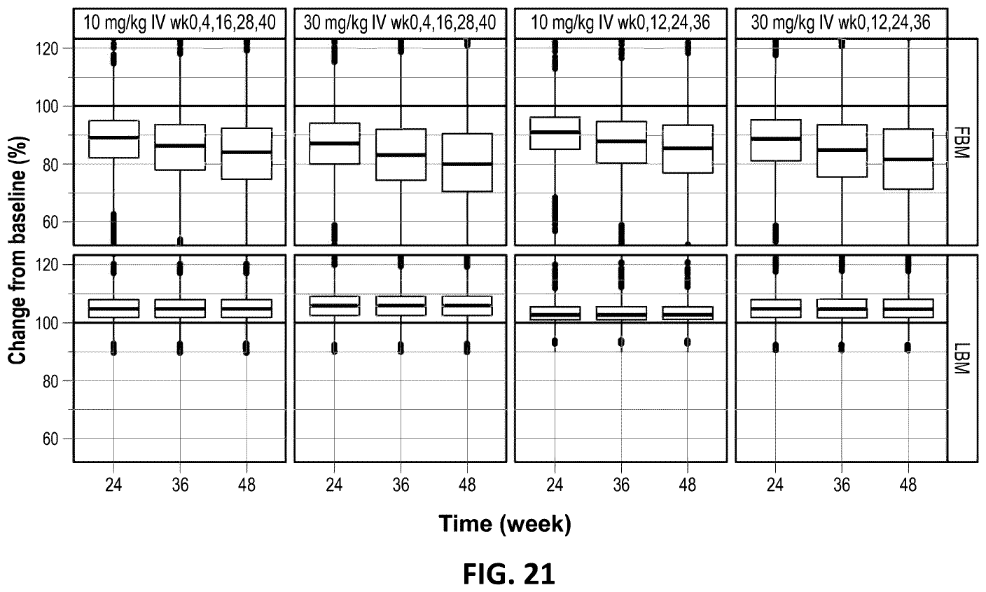

FIG. 19 shows the predicted effect on LBM and FBM as a percent (%) change from baseline. FIG. 20 shows the predicted effect of bimagrumab dosage on the FBM+LBM. Table 5 and FIG. 21 show the summary statistics for the predicted effect FBM and LBM effects of bimagrumab dosage at 24, 36, and 48 weeks.

TABLE 5
Predicted FBM and LBM response for bimagrumab dosage regimens
of every 12 weeks with the addition of a loading dose.

| | | FBM Median | LBM Median |
|---|---|---|---|
| | | (10th, 90th | (10th, 90th |
| Dosing | Week | percentile) | percentile) |
| 10 mg/kg IV | 24 | 89 (75.4, 89) | 104.8 (99.92, 104.8) |
| wk 0, 4, 16, 28, 40 | 36 | 86.2 (70.4, 86.2) | 104.7 (99.92, 104.7) |
| | 48 | 84 (66.9, 84) | 104.6 (99.92, 104.6) |
| 30 mg/kg IV | 24 | 87.1 (72.2, 87.1) | 105.9 (100, 105.9) |
| wk 0, 4, 16, 28, 40 | 36 | 83 (66.1, 83) | 105.9 (100, 105.9) |
| | 48 | 79.8 (61.7, 79.8) | 105.9 (100, 105.9) |
| 10 mg/kg IV | 24 | 90.9 (78.5, 90.9) | 102.7 (99.89, 102.7) |
| wk 0, 12, 24, 36 | 36 | 87.7 (72.8, 87.7) | 102.7 (99.89, 102.7) |
| | 48 | 85.3 (68.6, 85.3) | 102.7 (99.89, 102.7) |
| 30 mg/kg IV | 24 | 88.6 (73.8, 88.6) | 104.6 (99.68, 104.6) |
| wk 0, 12, 24, 36 | 36 | 84.6 (67.1, 84.6) | 104.7 (99.68, 104.7) |
| | 48 | 81.5 (62.7, 81.5) | 104.7 (99.68, 104.7) |

### Predicted Effect of Body Weight on Bimagrumab PK and PD

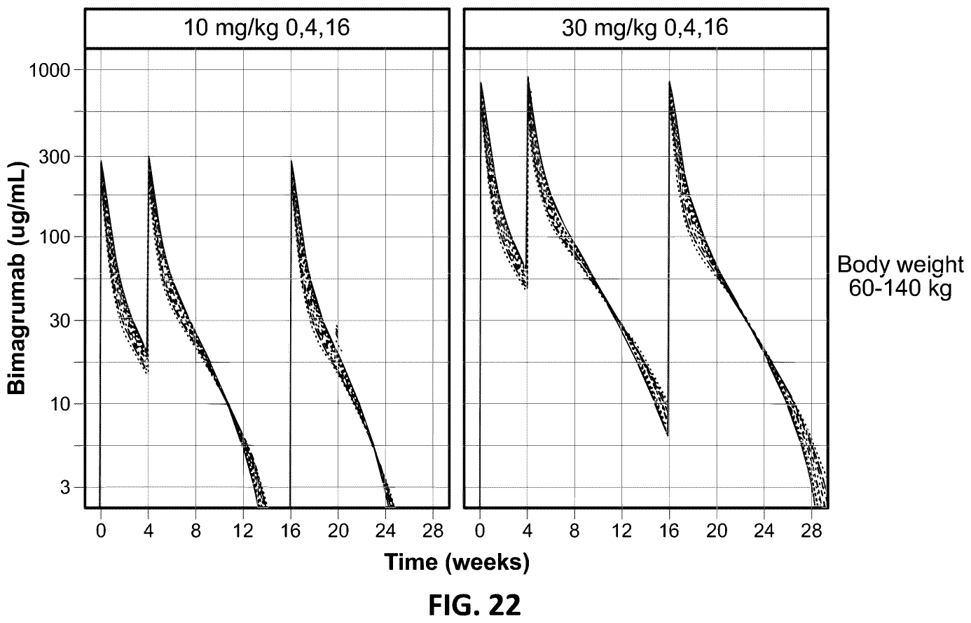

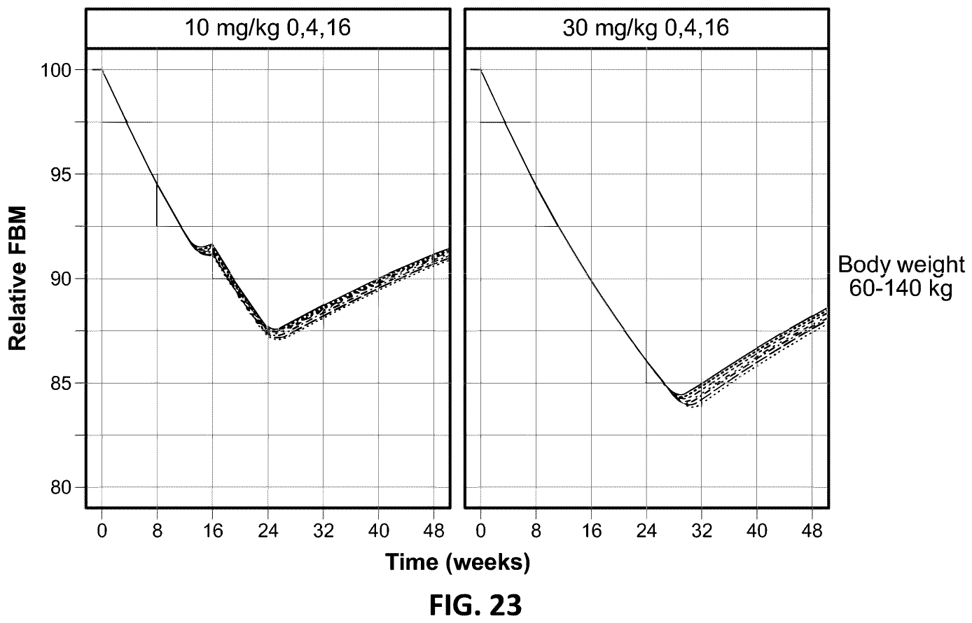

PK/PD simulations were run to assess the effect of body weight on bimagrumab dosing PK and PD effects. Subjects with population-typical parameters and bodyweights of 60 to 140 kg were simulated. FIG. 22 and FIG. 23 show the bimagrumab concentration versus time profiles (PK) and the effects on FBM (PD), respectively. The peak serum concentration of bimagrumab (Cmax) and the 24 week area under the curve (AUC) were computed from the bimagrumab exposure per body weight and are summarized in Tables 6 and 7.

TABLE 6
Effect of body weight on 24 week AUC.

| Treatment | 60 | 70 | 80 | 90 | 100 | 110 | 120 | 130 | 140 |
|---|---|---|---|---|---|---|---|---|---|
| 10 mg/kg 0, 4, 16 | 4′790 | 5′148 | 5′475 | 5′777 | 6′059 | 6′324 | 6′574 | 6′811 | 7′037 |
| 30 mg/kg 0, 4, 16 | 15′889 | 16′976 | 17′967 | 18′881 | 19′732 | 20′530 | 21′283 | 21′997 | 22′677 |

TABLE 7
Effect of body weight on Cmax

| Treatment | 60 | 70 | 80 | 90 | 100 | 110 | 120 | 130 | 140 |
|---|---|---|---|---|---|---|---|---|---|
| 10 mg/kg 0, 4, 16 | 210 | 222 | 232 | 241 | 250 | 258 | 266 | 274 | 280 |
| 30 mg/kg 0, 4, 16 | 631 | 665 | 696 | 724 | 751 | 775 | 799 | 821 | 841 |

The predicted median effect on FBM at week 48 in the bimagrumab low dose regimen with a loading dose (10 mg/kg given IV at 0, 4, 16, 28, and 40 weeks) and in the bimagrumab high dose regimen with a loading dose (30 mg/kg given IV at 0, 4, 16, 28, and 40 weeks) were 84 and 79.8 of the FBM baseline and 104.6 and 105.9 of the LBM baseline.

PK/PD simulations showed that the effect on LBM was much faster and that the steady state was obtained within three months, while the time to steady state for FBM took at least two years. Notably, because of the different effect kinetics in FBM and LBM, it was predicted that after start of treatment with bimagrumab, patients first gain in body mass for about 2-3 months after which they start to lose body mass compared to baseline due to the larger effect size on FBM. After cessation of treatment, the effect on LBM was lost within about 3 months, while the effect on FBM took about one year to return to half of the effect size.

Notably, the addition of the loading dose to the dosage regimen of 30 mg/kg maintains the C_{trough} above 10 μg/ml (as shown in FIG. 18), which is predicted to be the saturation concentration of the ActRII receptor (FIG. 24). Thus, the dosage regimen of 30 mg/kg every 12 weeks with at least one loading dose is predicted to enable continuous fat loss.

### Example 6: Assessment of Bimagrumab Dosage in Adults Who are Overweight or Obese

A randomized, placebo-controlled study was designed to assess bimagrumab dosage in adults 45 and older who are overweight or obese. The purpose of the study is to assess the effect of bimagrumab on fat loss in adults ages 45 and above who are obese or who are overweight with at least one obesity related co-morbidity.

The bimagrumab dosage regimens compared herein include a high dose regimen (30 mg/kg) and a low dose regimen (10 mg/kg) administered every 12 weeks. Both bimagrumab dosage regimens are preceded by a loading dose (at week 0) 4 weeks prior to the start of the dosage regimen.

### Primary Objective

To determine safe and efficacious bimagrumab dosage with respect to central adiposity and fat mass loss, as measured by a change in waist circumference (cm) from baseline at Week 0 to Week 24.

### Secondary Objectives

I. To assess the safety and tolerability of bimagrumab dosage.
II. To evaluate the effect of bimagrumab with respect to change in lean mass (kg and % change) from baseline at Week 0 to Week 24, as measured by DXA.
III. To assess if bimagrumab improves muscle strength as measured by hand dynamometry from baseline at Week 0 to Week 24.
IV. To evaluate the effect of bimagrumab with respect to change in fat mass (kg and % change) from baseline at Week 0 to Week 24, as measured by DXA.
V. To assess change from baseline at Week 0 to Week 24 in body weight (kg) and body composition as measured by bioelectrical impedance analysis (BIA) for each treatment group.
VI. To assess the proportion of participants in each treatment group experiencing≥5% body weight reduction from baseline at week 0 to Week 24 (%).
VII. To assess change from baseline at Week 0 to Week 24 in systolic and diastolic blood pressure (mmHg) for each treatment group.
VIII. To assess change from baseline at Week 0 to Week 24 in HbA1C and HOMA2 for each treatment group
IX. To determine the change of all the objective primary and secondary endpoints above from week 24 to week 36
X. To assess change from baseline at Week 0 to Week 24 in participants health status (SF-36)
XI. To assess change from baseline at Week 0 to Week 24 in participants quality of life (IWQoL-Lite for CT)
XII. To evaluate the effect of bimagrumab using a change in bioelectrical impedance (percent Ohms change) from the baseline at Week 0 to Week 24.
XIII. To determine the change in all primary and secondary objectives from week 24 to week 36.

### Efficacy Endpoints

To determine the efficacy of bimagrumab with respect to fat mass loss, the following markers are assessed: waist circumference; blood pressure; fat mass by dual energy X-ray absorptiometry (DXA); lean mass by DXA; body weight; body mass index (BMI); lipid levels (total cholesterol (TC), high-density lipoprotein (HDL), low-density lipoprotein (LDL), and trigylcerides (TG)); HgbA1c levels; glucose levels; insulin levels; short physical performance battery (SPPB); physical activity monitoring via actigraphy; the Impact of Weight on Quality of Life (IWQoL-Lite for CT), the Short Form (36) Health Survey (SF-36), exploratory urine biomarkers (e.g., microalbumin); leptin, adiponectin, adipsin, IL-18, and IL-6 as adipose tissue biomarkers, and Activin A as a pharmacodynamic (PD) biomarker.

### Safety Endpoints

To determine the safety of the treatment, clinically determined adverse events (AEs) are monitored. These include but are not limited to treatment emergent adverse events (TEAEs) and serious adverse events (SAEs). Further, the change from baseline of patient health markers from week 0 to week 24 are monitored, including: heart rate (bpm), amylase (U/L), lipase (U/L), Alkaline Phosphatase ALP (U/L), aspartate aminotransferase AST (U/L), and alanine aminotransferase ALT (U/L).

### Study Design

The study is randomized, partially blinded, and placebo-controlled, with a 3-arm trial. Participants are randomized to one of the following treatment arms, including a loading dose:
a) Bimagrumab placebo
b) Bimagrumab 10 mg/kg
c) Bimagrumab 30 mg/kg

Bimagrumab and bimagrumab placebo treatment are kept blinded. The study consists of a screening visit to assess eligibility, followed by visits and phone calls every 4 weeks during the treatment phase, until the follow up at week 24. The End of Study visit is performed at week 36.

### Investigational Product Dosage and Mode of Administration

As shown above in the study design, bimagrumab is given in a dose of either 10 mg/kg or 30 mg/kg each dose by intravenous (IV) infusion, at week 0 (loading dose), week 4, and week 16. This dosage regimen is every 12 weeks with the addition of a loading dose at week 0 prior to the start of the dosage regimen at week 4.

The rationale for a 24 week exposure duration and primary endpoint assessment is that this duration allows time for observation of differential effects from the bimagrumab doses relative to the placebo.

### Reference Treatment Dosage and Mode of Administration

The bimagrumab placebo includes dextrose 5% in water (D5W) and is administered by IV infusion in 2 doses, one dose at week 0 and the second dose at week 12.

### Duration of Study

The study is up to 40 weeks in duration, including a 4-week screening period, a 24-week treatment period, and a 12 week follow up period.

An alternative study paradigm includes a 48 week treatment period, wherein bimagrumab is given in a dose of either 10 mg/kg or 30 mg/kg each dose by intravenous (IV) infusion, at week 0 (loading dose), week 4, week 16, week 28, and week 40.

### Inclusion Criteria

1. A written informed consent must be obtained before any study-related assessment.
2. Men and women aged≥45.
3. BMI≥30 or BMI≥27 with one or more obesity-associated comorbidities.
4. Have a stable body weight (±3 kg) within three months of screening
5. Have a history of at least 1 self-reported unsuccessful dietary effort to lose body weight
6. Able to communicate well with the investigator, comply with the study requirements and adhere to the diet and activity programs for the study duration.

### Exclusion Criteria

1. History of a severe reaction to a monoclonal antibody treatment
2. Use of other investigational drugs at the time of enrollment or within 30 days or 5 half-lives of enrollment, whichever is longer, or longer if required by any other limitation of participation in an investigational trial based on local regulations.
3. Patient weight>150 kg
4. History of clinically significant condition, for example cardiovascular or pulmonary disease or osteoarthritis, that precludes regular walking for exercise
5. Contraindication to following a 500 calorie daily deficit, high protein diet
6. Pregnant or nursing women.
7. Women of child-bearing potential, defined as all women physiologically capable of becoming pregnant, UNLESS they are using highly effective methods of contraception during dosing and for 6 months after the last dose of bimagrumab.

### Highly Effective Contraception Methods Include:

1. Total abstinence. Periodic abstinence (e.g., calendar, ovulation, symptothermal, post-ovulation methods) and withdrawal are not acceptable methods of contraception.
2. Female sterilization (surgical bilateral oophorectomy with or without hysterectomy) or tubal ligation at least 6 weeks before taking study treatment. In case of oophorectomy alone, only when the reproductive status of the woman has been confirmed by follow up hormone level assessment.
3. Male sterilization (at least 6 months prior to screening). For female participants in the study, the vasectomized male partner should be the sole partner for that participant.
4. Combination of any two of the following methods (a+b or a+c or b+c):
a) Oral, injected or implanted hormonal methods of contraception or other forms of hormonal contraception with comparable efficacy (failure rate<1%), e.g., hormone vaginal ring or transdermal hormone contraception.
b) Intrauterine device or intrauterine system.
c) Barrier methods of contraception e.g., condom or occlusive cap (diaphragm or cervical/vault caps) with spermicidal foam/gel/film/cream/vaginal suppository.

In case of use of oral contraception women should have been stable on the same pill for a minimum of 3 months before taking study treatment.

Women are considered post-menopausal and not of childbearing potential if they have had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least six weeks ago. In the case of oophorectomy alone, only when the reproductive status of the woman has been confirmed by follow up hormone level assessment is the woman considered not of childbearing potential.
8. History of malignancy of any organ system, treated or untreated within the past five years, regardless of whether there was evidence of local recurrence or metastases, except non-melanoma skin cancer treated with local therapy, prostate cancer managed with watchful waiting, or breast or cervical carcinoma treated with local excision alone.
9. Personal or family history of medullary thyroid carcinoma or in patients with Multiple Endocrine Neoplasia syndrome type 2.
10. Disease other than cancer known to cause cachexia or muscle atrophy, disease known to cause GI malabsorption disease associated with lipodystrophy.
11. Diagnosis of diabetes plus current use of any anti-diabetic drug. Metabolic syndrome is not an exclusion, even if managed with an anti-diabetic drug such as metformin or an SGLT2 inhibitor.
12. Uncontrolled hypothyroidism. Hypothyroid patients treated with hormone replacement therapy must be on a stable dose for at least 6 weeks prior to the Screening visit.
13. Significant psychiatric disorder, clinically manifest peripheral vascular disease or disorder, or systemic disorder which could affect any of the efficacy assessments (e.g., diabetic neuropathy, chronic fatigue syndrome, schizophrenia, bipolar disorder, severe depression, intermittent claudication).
14. Known heart failure classified as New York Heart Association Class III and IV or a history of chronic hypotension (systolic blood pressure<100 mmHg).
15. Systolic blood pressure>180 or <90 mm Hg or diastolic blood pressure>100 or <50 mmHg at screening, or malignant hypertension.
16. ECG showing clinically significant abnormalities including any current supra-ventricular arrhythmia with an uncontrolled ventricular response (mean heart rate>100 beats per minute [bpm]) at rest despite medical or device therapy, or any history of spontaneous or induced sustained ventricular tachycardia (heart rate>100 bpm for 30 sec) despite medical or device therapy, or any history of resuscitated cardiac arrest or presence of an automated internal cardioverter-defibrillator.
17. History of unstable angina, myocardial infarction, coronary artery bypass graft surgery, or percutaneous coronary intervention (such as angioplasty or stent placement) within 180 days of the Screening visit.
18. Prolonged QT syndrome or QTcF>450 msec (Fridericia Correction) for males and >470 msec for females at screening or baseline at repeated assessment.
19. Significant coagulopathy, platelet count less than 75,000/mm³, hemoglobin less than 11.0 g/dL.
20. Liver disease or liver injury as indicated by abnormal liver function tests such as SGOT (AST), SGPT (ALT), alkaline phosphatase, or serum bilirubin (except Gilbert's Disease). The Investigator should be guided by the following criteria:

Any single transaminase may not exceed 3× times upper limit of normal (ULN). A single parameter elevated up to and including 3×ULN should be re-checked as soon as possible, and in all cases, at least prior to randomization, to rule out any lab error.

If the total bilirubin concentration is increased above 1.5×ULN, total bilirubin should be differentiated into the direct and indirect reacting bilirubin. In any case, serum bilirubin should not exceed the value of 1.6 mg/dL (27 μmol/L).
21. Known history or presence of severe acute or chronic liver disease (compensated or decompensated), known gallbladder or bile duct disease, acute or chronic pancreatitis, acute renal failure or chronic renal failure stage 3 or worse.
22. History of hepatitis B or HIV. History of hepatitis A or hepatitis C successfully treated is not exclusionary.
23. Use of any prescription drugs known to affect muscle mass adversely, including anti-androgens (such as luteinizing hormone releasing hormone (LHRH) agonists), anti-estrogens (tamoxifen, etc.), etc. Low dose estrogen replacement therapy in post-menopausal women is acceptable and 5-alpha reductase inhibitors in men are acceptable.
24. Lack of peripheral venous access.
25. Donation or loss of 400 mL or more of blood within eight weeks prior to a loading dose, or longer if required by local regulation, or plasma donation (>250 mL) within 14 days prior to the first dose.
26. Acute illness within the 30 days prior to the screening visit that, in the opinion of the investigator, affects lower extremity function or the patient's ability to participate in the study.
27. Smoking more than one pack of cigarettes daily.
28. Using cannabis more than twice weekly.
29. Drinking 5 or more alcoholic beverages on the same occasion on each of 5 or more days in the past 30 days.
30. A familial history of hypertriglyceridemia or history of fasting triglyceride greater than 500 mg/dl (5.65 mmol/L).

1. A method of treating a metabolic disorder in a subject in need thereof, comprising administering to the subject an ActRII antibody dosage regimen, wherein the ActRII antibody dosage regimen comprises administration of an ActRII antibody about once every 8 weeks to about once every 16 weeks, in a dose of about 3 mg/kg to about 50 mg/kg, wherein the ActRII antibody dosage regimen is administered intravenously.

2. The method of claim 1, wherein the ActRII antibody dosage regimen follows the administration of an ActRII antibody loading dose.

3. The method of claim 2, wherein the ActRII antibody dosage regimen and/or the ActRII antibody loading dose comprises administration of an ActRII antibody comprising the amino acid sequence of SEQ ID NOS: 1-6.

4. The method of claim 1, wherein the ActRII antibody dosage regimen and/or the ActRII antibody loading dose comprises administration of an ActRII antibody comprising: the amino acid sequence of SEQ ID NO: 7, or a sequence with sequence identity of at least 90% thereto, and comprising the amino acid sequence of SEQ ID NO: 8, or a sequence with sequence identity of at least 90% thereto.

5. The method of claim 1, wherein the ActRII antibody dosage regimen and/or the ActRII antibody loading dose comprises administration of an ActRII antibody comprising: the amino acid sequence of SEQ ID NO: 9, or a sequence with sequence identity of at least 90% thereto, and comprising the amino acid sequence of SEQ ID NO: 10, or a sequence with sequence identity of at least 90% thereto.

6. The method of claim 1, wherein the ActRII antibody dosage regimen comprises administration of an ActRII antibody specific for ActRIIA and ActRIIB.

7. The method of claim 1, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

8. The method of claim 7, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

9. The method of claim 1, wherein the metabolic disorder is Prader-Willi syndrome.

10. The method of claim 7, wherein the diabetes is Type I diabetes or Type II diabetes.

11. The method of claim 7, wherein the method treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidemia, hypertension, heart failure, coronary heart disease, stroke, and/or gallstones.

12. The method of claim 1, wherein the treatment reduces body weight in the subject.

13. The method of claim 1, wherein the treatment reduces fat mass in the subject.

14. The method of claim 1, wherein the treatment increases lean mass in the subject.

15. The method of claim 1, wherein the treatment reduces fat mass and increases lean mass in the subject.

16. The method of claim 1, wherein the treatment reduces fat mass and maintains lean mass in the subject.

17. The method of claim 1, wherein the treatment reduces waist circumference in the subject.

18. The method of claim 1, wherein the treatment reduces liver and/or non-liver fat mass in the subject.

19. The method of claim 1, wherein the treatment improves glycemic control in the subject.

20. The method of claim 1, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

21. The method of claim 1, wherein the efficacy of the treatment is measured by the C_{trough} in a sample from the subject.

22. The method of claim 21, wherein the treatment is efficacious when the C_{trough} of the sample from the subject is at least about 500%, about 400%, about 300%, about 200%, about 100%, about 90%, about 80%, or about 75% of a desired “C_{trough}”.

23. The method of claim 22, wherein the desired “C_{trough}” is 10 μg/ml.

24. The method of claim 1, wherein the subject is human.

25. A method of treating a metabolic disorder in a subject in need thereof comprising administering to the subject an ActRII antibody loading dose followed by an ActRII antibody dosage regimen.

26. The method of claim 25, wherein the ActRII antibody loading dose is about 3 mg/kg to about 50 mg/kg.

27. The method of claim 25, wherein the ActRII antibody dosage regimen starts about 1 day, about 2 days, about 3 days, about 4 days, about 5 days, about 6 days, about 1 week, about 2 weeks, about 3 weeks, about 4 weeks, or about 5 weeks after the loading dose of the ActRII antibody.

28. The method of claim 25, wherein the ActRII antibody dosage regimen comprises administration of an ActRII antibody in a dose of about 3 mg/kg to about 50 mg/kg.

29. The method of claim 25, wherein the ActRII antibody dosage regimen comprises administration of an ActRII antibody about every 4 weeks, about every 8 weeks, about every 12 weeks, or about every 16 weeks.

30. The method of claim 25, wherein the ActRII antibody loading dose is administered intravenously.

31. The method of claim 25, wherein the ActRII antibody of the loading dose and/or the ActRII antibody of the dosage regimen comprises the amino acid sequence of SEQ ID NOS: 1-6.

32. The method of claim 25, wherein the ActRII antibody of the loading dose and/or the ActRII antibody of the dosage regimen comprises the amino acid sequence of SEQ ID NO: 7, or a sequence with sequence identity of at least 90% thereto; and comprises the amino acid sequence of SEQ ID NO: 8, or a sequence with sequence identity of at least 90% thereto.

33. The method of claim 25, wherein the ActRII of the loading dose and/or the ActRII antibody of the dosage regimen comprises the amino acid sequence of SEQ ID NO: 9, or a sequence with sequence identity of at least 90% thereto; and comprises the amino acid sequence of SEQ ID NO: 10, or a sequence with sequence identity of at least 90% thereto.

34. The method of claim 25, wherein the ActRII antibody of the loading dose and/or the ActRII antibody of the dosage regimen is specific for ActRIIA and ActRIIB.

35. The method of claim 25, wherein the metabolic disorder is selected from the group consisting of: obesity, diabetes, metabolic syndrome, anti-psychotic drug-associated obesity, glucocorticoid-induced obesity, hypothalamic obesity associated with craniopharyngioma, and a monogenetic disorder associated with obesity.

36. The method of claim 35, wherein the monogenetic disorder associated with obesity, is one of Bardet-Biedl syndrome, or obesity resulting from mutations in one or more of the genes comprising: ADCY3, ALMS1, ARL6, BBS1, BBS2, BBS4, BBS5, BBS7, BBS9, BBS10, BBS12, BDNF, CCDC28B, CEP290, CREBBP, EP300, GNAS, IER3IP1, MKKS, MKS1, MRAP2, NTRK2, PCSK1, PHF6, POMC, SH2B1, SIM1, TMEM67, TRIM32, TTC8 and VPS13B.

37. The method of claim 25, wherein the metabolic disorder is Prader-Willi syndrome.

38. The method of claim 35, wherein the diabetes is Type I diabetes or Type II diabetes.

39. The method of claim 35, wherein the method treats an obesity related co-morbidity, selected from the group of: glucose intolerance, prediabetes, insulin resistance, high triglycerides, overweight associated physical impairment, osteoporosis, renal disease, cardiometabolic disease, non-alcoholic fatty liver disease, obstructive sleep apnea, sexual hormones impairment, endocrine reproductive disorders, osteoarthritis, gastrointestinal cancers, dyslipidaemia, hypertension, heart failure, coronary heart disease, stroke, and/or gallstones.

40. The method of claim 25, wherein the treatment reduces body weight in the subject.

41. The method of claim 25, wherein the treatment reduces fat mass in the subject.

42. The method of claim 25, wherein the treatment increases lean mass in the subject.

43. The method of claim 25, wherein the treatment reduces fat mass and increases lean mass in the subject.

44. The method of claim 25, wherein the treatment reduces fat mass and maintains lean mass in the subject.

45. The method of claim 25, wherein the treatment reduces waist circumference in the subject.

46. The method of claim 25, wherein the treatment reduces liver and/or non-liver fat mass in the subject.

47. The method of claim 25, wherein the treatment improves glycemic control in the subject.

48. The method of claim 25, wherein the efficacy of the treatment is measured by at least one of the following: body weight; bioelectrical impedance analysis (BIA); dual X-ray absorptiometry (DXA); magnetic resonance imaging (MRI); waist circumference; decreased BMI; waist to hip ratio; wait to height ratio; blood lipids profile; leptin, adiponectin, and adipsin levels; urine biomarkers; hemoglobin A1c (HgbA1c) levels; hand dynamometry demonstrating muscle strength; glucose levels; insulin levels; short physical performance battery (SPPB); Impact of Weight on Quality of Life (IWQoL-Lite for CT) assessment; Short Form (36) Health Survey (SF-36) assessment; homeostasis model assessment 2 (HOMA2); and physical activity monitoring via actigraphy.

49. The method of claim 25, wherein the efficacy of the treatment is measured by the C_{trough} in a sample from the subject.

50. The method of claim 49, wherein the treatment is efficacious when the C_{trough} of the sample from the subject is at least about 500%, about 400%, about 300%, about 200%, about 100%, about 90%, about 80%, or about 75% of a desired “C_{trough}”.

51. The method of claim 50, wherein the desired “C_{trough}” is 10 μg/ml.

52. The method of claim 25, wherein the subject is human.


SEQUENCE LISTING (10 sequences)

SEQ ID NO: 1 - protein, 10 aa, synthetic construct
GYTFTSSYIN

SEQ ID NO: 2 - protein, 16 aa, synthetic construct
TINPVSGSTSYAQKFQ

SEQ ID NO: 3 - protein, 6 aa, synthetic construct
GGWFDY

SEQ ID NO: 4 - protein, 14 aa, synthetic construct
TGTSSDVGSYNYVN

SEQ ID NO: 5 - protein, 10 aa, synthetic construct
MIYGVSKRPS

SEQ ID NO: 6 - protein, 10 aa, synthetic construct
GTFAGGSYYG

SEQ ID NO: 7 - protein, 115 aa, synthetic construct [sequence omitted]

SEQ ID NO: 8 - protein, 113 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQ

SEQ ID NO: 9 - protein, 445 aa, synthetic construct
QVQLVQSGAEVKKPGASVKVSCKASGYTFTSSYINWVRQAPGQGLEWMGTINPVSGSTSYAQKFQGRVTMTRDTSISTAYMELSRLRSDDTAVYYCARGGWFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, synthetic construct
QSALTQPASVSGSPGQSITISCTGTSSDVGSYNYVNWYQQHPGKAPKLMIYGVSKRPSGVSNRFSGSKSGNTASLTISGLQAEDEADYYCGTFAGGSYYGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS